(19) United States
(12) Patent Application Publication
(10) Pub. No.: US 2025/0084116 A1
(43) Pub. Date: Mar. 13, 2025

(54) MIXTURE OF STEREOISOMERS OF A SULFATED GLYCOLIPID

(71) Applicant: NATIONAL RESEARCH COUNCIL OF CANADA, Ottawa (CA)

(72) Inventors: Usha HEMRAZ, Candiac (CA); Sophie RÉGNIER, Montreal (CA); Edmond LAM, Montreal (CA); Vinicio VASQUEZ, Laval (CA); Michael MCCLUSKIE, Ottawa (CA); Felicity STARK, Ottawa (CA); Bassel AKACHE, Ottawa (CA)

(21) Appl. No.: 18/724,432

(22) PCT Filed: Dec. 21, 2022
(86) PCT No.: PCT/IB2022/062644

§ 371 Date: Jun. 26, 2024

Related U.S. Application Data
(60) Provisional application No. 63/296,648, filed on Jan. 5, 2022.

(51) Int. Cl. C07H 15/06; A61K 39/00; A61K 39/39; C07C 41/09

(52) U.S. Cl. CPC C07H 15/06; A61K 39/39; C07C 41/09; A61K 2039/55555

(57) ABSTRACT

A composition comprising a mixture of two or more stereoisomers of a synthetic charged isoprenoid glycolipid of Formula (I), or a pharmaceutically acceptable salt thereof, in which n is 0 or 1, R and R′ are independently hydrogen or hydroxyl, each Y is independently hydrogen or a sulfate group, and wherein at least one Y is a sulfate group, and less than 25% of the synthetic charged isoprenoid glycolipid molecules in the mixture comprise an archaeol moiety of the configuration (R)-2,3-bis(((3R,7R,11R)-3,7,11,15-tetramethylhexadecyl)oxy) propan-1-ol. Further provided are archaeosomes and immunogenic compositions comprising the composition, use of the composition as an adjuvant or immunostimulant, and processes for synthesizing the composition.

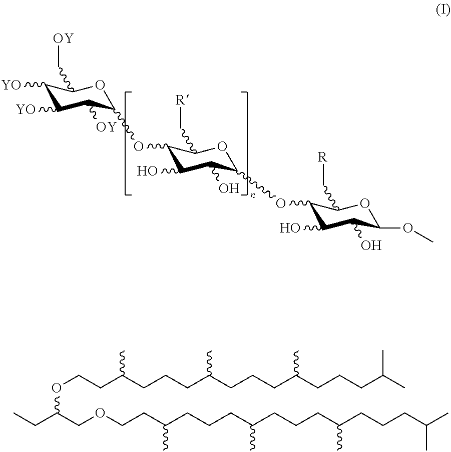

### FIELD

The present disclosure relates to charged glycolipid compositions, particularly to charged glycolipid compositions of mixed stereochemistry, and formulations thereof that can be used to prepare archaeosomes and other lipid compositions that are useful as adjuvants.

### BACKGROUND

Archaeosomes are a type of liposomes made of total polar or semi-synthetic lipids derived from archaea.¹ Archaeosome membrane lipids consists of branched, fully saturated phytanyl chains attached at the sn-2,3-glycerol carbons via ether bonds. These structural features may be responsible for the high pH stability and thermal stability of archaeosomes, as well as their resistance towards lipase hydrolysis.¹ Archaeosomes are often used as drug delivery systems,² in particular for vaccine antigens, due to their strong immunostimulatory properties.³

The importance of stereochemistry in active pharmaceutical ingredients has been explored as far back as the nineteenth century. Although two stereoisomers may have the exact same molecular formula and atom-to-atom linkage, they cannot be superimposed and therefore can be recognized differently in a biological system.⁵ This difference in biological recognition can cause severe consequences, as was observed with the drug thalidomide when it was first marketed as a racemate of R and S enantiomers. Although the R enantiomer proved to have good sedative effects, the S enantiomer interfered with vasculogenesis, creating malformations in foetuses.⁶ The tragedy of thalidomide resulted in tighter regulations in drug development to increase the safety associated with drug administration. The absolute stereochemistry of the basic structural unit of the archaeol (R)-2,3-bis(((3R,7R,11R)-3,7,11,15-tetramethylhexadecyl)oxy) propan-1-ol (1) (FIG. 1) is well-defined.⁴

As essential components of some vaccines, adjuvants enhance vaccine efficacy by triggering strong and long-lasting antigen-specific immune responses.⁸ In recent years, archaeosomes composed of a single sulfated lactosyl archaeol (SLA) glycolipid, namely, 6′-sulfate-β-D-Galp-(1,4)-β-D-Glcp-(1,1)-archaeol (SLA-1, FIG. 1) have emerged as a potential adjuvant in preclinical studies.⁹⁻¹⁰ The immunostimulatory effects of SLA archaeosomes as an adjuvant in formulations with ovalbumin (Ova) and hepatitis B surface antigen (HBsAg) showed superior adjuvanticity¹¹ and strong antigen-specific immune responses¹² compared to commercial adjuvants, including TLR3/4/9 agonists, oil-in-water and water-in-oil emulsions and aluminum hydroxide.¹¹ SLA formulations have also been evaluated as an adjuvant to the H1N1 influenza hemagglutinin (HA) protein in a murine model and strong anti-HA responses were observed in young, aged and pregnant mice. For the latter, protection against subsequent viral infections was also extended to the pups through the immunized mothers.¹³ In another study, it was found that co-administration of SLA archaeosomes with other adjuvants enhanced both humoral and cellular immune responses compared to each of the adjuvants alone.¹⁴ This ability to induce long-lasting humoral and cell-mediated immune responses prompted an evaluation of SLA's abilities to enhance the immunogenicity of a synthetic long peptide for potential applications in oncology. It was found that in combination with Poly(I:C), strong robust antigen-specific CD8+ T cell responses were induced and this formulation was also effective at suppressing tumor growth and extended mouse survival in a mouse melanoma tumor model.¹⁵ The mechanism through which SLA attains such strong adjuvanticity is not known.

SLA has been produced in a semi-synthetic fashion, with the glycosyl moiety coming from chemically modified lactose, and the archaeol moiety coming from archaea growth and extraction.¹⁶⁻¹⁸ When SLA is produced in this manner, all seven chiral centres of the archaeol moiety are in the R configuration. While the semi-synthetic procedure yields SLA in high yields,¹⁶ the microbial growth and subsequent extraction and purification steps to produce the archaeol are time-consuming. A fully synthetic process to produce archaeol would be faster and more scalable than producing archaeol from archaea.

A 5-step enantioselective synthesis of (R)-2,3-bis(((3R,7R,11R)-3,7,11,15-tetramethylhexadecyl)oxy) propan-1-ol (1) has been described in the literature⁴ and requires 3-O-benzyl-sn-glycerol, which can be synthesized in five steps from commercially available mannitol.¹⁹ However, this process is laborious and time consuming.

### SUMMARY

The present inventors have developed a synthetic process for producing a mixture of two or more stereoisomers of a synthetic charged isoprenoid glycolipid as described herein. The synthetic charged isoprenoid glycolipid may be used as an adjuvant in an immunogenic composition, such as a vaccine composition, to enhance or direct an immune response to an antigen.

Accordingly, there is provided a composition comprising a mixture of two or more stereoisomers of:
a synthetic charged isoprenoid glycolipid comprising a sulfated saccharide group covalently linked to the free sn-1 hydroxyl group of the glycerol backbone of an archaeol moiety via a beta linkage, wherein the synthetic charged glycolipid is a compound of the formula:

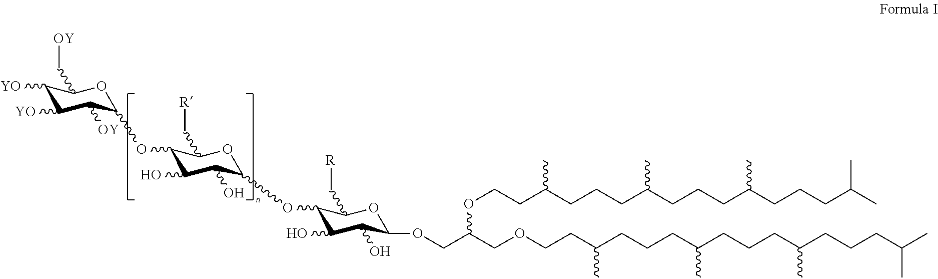

or a pharmaceutically acceptable salt thereof,
wherein
n is 0 or 1;
R and R′ are independently hydrogen or hydroxyl;
each Y is independently hydrogen or a sulfate group, and wherein at least one Y is a sulfate group; and
less than 25% of the synthetic charged isoprenoid glycolipid molecules in the mixture comprise an archaeol moiety of the configuration (R)-2,3-bis(((3R,7R,11R)-3,7,11,15-tetramethylhexadecyl)oxy) propan-1-ol. In embodiments, less than 10%, about 5% to about 8%, or about 6.5% of the synthetic charged isoprenoid glycolipid molecules in the mixture comprise an archaeol moiety of the configuration (R)-2,3-bis(((3R,7R,11R)-3,7,11,15-tetramethylhexadecyl)oxy) propan-1-ol.

In an embodiment, only one Y is a sulfate group.

In an embodiment, the sulfated saccharide group comprises monosaccharide moieties selected from the group consisting of mannose (Man), glucose (Glc), rhamnose (Rha), and galactose (Gal) moieties. In a further embodiment, the compound comprises a sulfate group at the 6′ position of the terminal monosaccharide moiety.

In an embodiment, n is 0 and R is OH.

In an embodiment, the synthetic charged isoprenoid glycolipid is 6′-sulfate-α-D-Manₚ-(1,6)-β-D-Galₚ-(1,4)-β-D-Glcₚ-(1,1)-archaeol, or 6′-sulfate-β-D-Glcₚ-(1,6)-β-D-Galₚ-(1,4)-β-D-Glcₚ-(1,1)-archaeol, or 6′-sulfate-β-D-Galₚ-(1,4)-β-D-Glcₚ-(1,6)-β-D-Glcₚ-(1,1)-archaeol, or a pharmaceutically acceptable salt thereof.

In an embodiment, the sulfated saccharide group is a sulfated lactosyl group. In an embodiment, the sulfated lactosyl group is a 6′-S-lactosyl group. In an embodiment, the 6′-S-lactosyl group is 6′-sulfate-β-D-Galₚ-(1,4)-β-D-Glcₚ.

In an embodiment, the synthetic charged isoprenoid glycolipid is a compound of the structure:

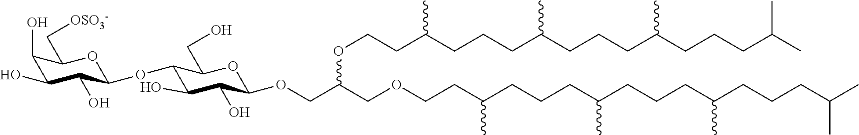

or a pharmaceutically acceptable salt thereof.

Another aspect of the disclosure is an archaeosome comprising a synthetic charged isoprenoid glycolipid composition as described herein.

Another aspect of the disclosure is an immunogenic composition comprising a synthetic charged isoprenoid glycolipid composition or archaeosome as described herein, and an antigen. In an embodiment, the antigen is a peptide, protein, or virus-like particle. In an embodiment, the immunogenic composition further comprises a pharmaceutically acceptable carrier. In an embodiment, the immunogenic composition further comprises an adjuvant other than a synthetic charged isoprenoid glycolipid. In an embodiment, the immunogenic composition is a vaccine composition.

Another aspect of the disclosure is a method of inducing an immune response in a subject, the method comprising administering an immunogenic composition as described herein to a subject.

Another aspect of the disclosure is a process for synthesizing a composition comprising a mixture of two or more stereoisomers of an archaeol, the process comprising treating (±)-3-benzyloxy-1,2-propanediol with a mesylated phytol derivative through a double nucleophilic substitution reaction, followed by a reductive debenzylation reaction,

wherein the archaeol is of the structure:

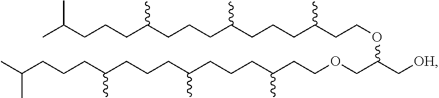

and wherein the mesylated phytol derivative is of the structure:

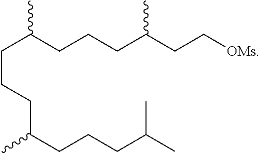

In an embodiment, the process comprises the following steps:

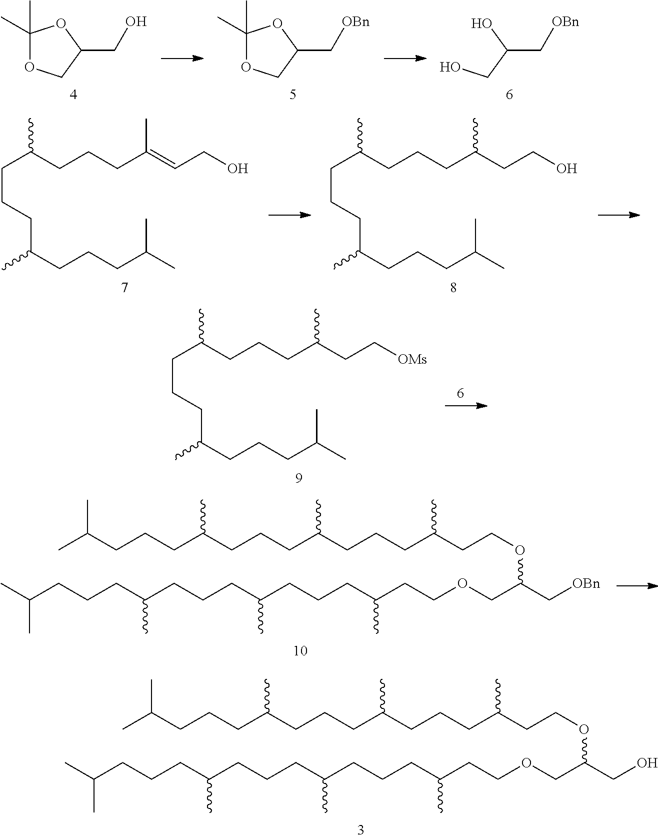

Another aspect of the disclosure is a process for synthesizing a mixture of two or more stereoisomers of a synthetic charged isoprenoid glycolipid or a pharmaceutically acceptable salt thereof, the process comprising covalently linking a sulfated saccharide group of the formula:

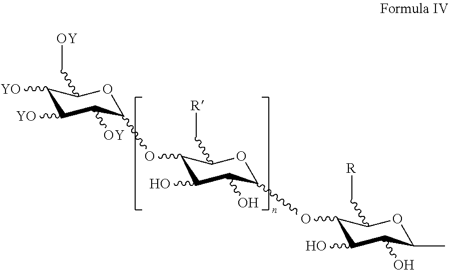

to the free sn-1 hydroxyl group of the glycerol backbone of an archaeol,
wherein the archaeol comprises a mixture of two or more stereoisomers of the structure:

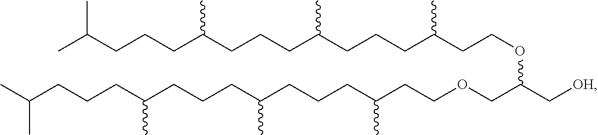

and wherein
n is 0 or 1;
R and R′ are independently hydrogen or hydroxyl;
each Y is independently hydrogen or a sulfate group, and wherein at least one Y is a sulfate group; and
less than 25% of the archaeol molecules in the mixture of two or more stereoisomers are of the configuration (R)-2,3-bis(((3R,7R,11R)-3,7,11,15-tetramethylhexadecyl)oxy) propan-1-ol. In embodiments, less than 10%, about 5% to about 8%, or about 6.5% of the archaeol molecules in the mixture of two or more stereoisomers are of the configuration (R)-2,3-bis(((3R,7R,11R)-3,7,11,15-tetramethylhexadecyl)oxy) propan-1-ol.

In an embodiment, the saccharide group is of the formula:

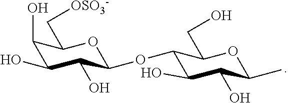

In an embodiment, the archaeol is produced according to a process as described herein.

Another aspect of the disclosure is use of a synthetic charged isoprenoid glycolipid composition or archaeosome as described herein for the manufacture of a vaccine or immunogenic composition.

Another aspect of the disclosure is use of a synthetic charged isoprenoid glycolipid composition or archaeosome as described herein as an adjuvant in a vaccine or immunogenic composition.

Another aspect of the disclosure is use of a synthetic charged isoprenoid glycolipid composition or archaeosome as described herein as an adjuvant to enhance or direct an immune response to an antigen in a subject.

Another aspect of the disclosure is a synthetic charged isoprenoid glycolipid composition or archaeosome as described herein, for use to enhance or direct an immune response to an antigen in a subject.

Another aspect of the disclosure is use of an immunogenic composition as described herein to induce an immune response to an antigen in a subject.

### BRIEF DESCRIPTION OF DRAWINGS

FIG. 1 shows structures of archaeols with varying chiral purities—100% of ((R)-2,3-bis(((3R,7R,11R)-3,7,11,15-tetramethylhexadecyl)oxy) propan-1-ol (1), mixture of 94% of ((R)-2,3-bis(((3R,7R,11R)-3,7,11,15-tetramethylhexadecyl)oxy) propan-1-ol and 6% of ((S)-2,3-bis(((3R,7R,11R)-3,7,11,15-tetramethylhexadecyl)oxy) propan-1-ol (2), and undefined 2,3-bis((3,7,11,15-tetramethylhexadecyl)oxy) propan-1-ol (3); and their corresponding SLAs—SLA-1, SLA-2 and SLA-3, respectively.

FIG. 2 shows a synthetic scheme for the non-stereoselective preparation of archaeol 3.

FIG. 3 shows a scheme for the synthesis of SLA-3 from lactose hydrate and archaeol 3.

FIG. 4 shows extracted ion chromatograms (m/z 653.65) of three archaeols: bacterial archaeol 1, a commercial archaeol purchased from Avanti 2, and synthesized archaeol 3.

FIG. 5 shows serum analysis of anti-Ova IgG titres for C57BL/6NCrl mice immunized once with the indicated vaccine formulations, with blood taken on day 20. Individual mouse titres (n=10) are represented with GMT±95% CI. A one-way ANOVA with Tukey's multiple comparison test was performed on the log-transformed values. Naive group is not shown, titres were <10.

FIG. 6 shows serum analysis of anti-Ova IgG titres for C57BL/6NCrl mice immunized twice (on days 0 and 21) with the indicated vaccine formulations, blood was taken on day 28. Individual mouse titres (n=10) are represented with GMT±95% CI. A one-way ANOVA with Tukey's multiple comparison test was performed on the log-transformed values. The following comparisons with MPL/alum were made with the dose 1 μg Ova and 1 mg SLA: ****SLA-1>MPL/alum-Ova **** SLA-2>MPL/alum-Ova; **** SLA-3>MPL/alum-Ova. Naive group is not shown, titres were <100.

FIG. 7 shows the number of IFN γ-secreting Ova-CD8⁺ T cells for C57BL/6NCrl mice immunized twice (on days 0 and 21) with the indicated vaccine formulations. IFN γ-secreting Ova-CD8⁺ T cells in the spleen were enumerated on day 28. Data (n=10) are shown as mean SEM and a one-wayANOVA with Tukey's multiple comparisons test was performed. The following comparisons were made with the dose 10 μg Ova and 1 mg SLA. **SLA-1>MPL/alum-Ova; **SLA-3>MPL/alum-Ova; *SLA-1>AddaVax™-Ova; *SLA-3>AddaVax™-Ova. Naïve group is not shown as there were less than 3 spots detected per mouse.

FIG. 8 shows the in vivo cytotoxicity of Ova-CD8+ T cells for C57BL/6NCrl mice immunized twice (days 0 and 21) with the indicated vaccine formulations. The in vivo cytotoxicity of Ova-CD8+ T cells was determined by injecting labelled target cells on day 27 and quantifying target cell killing among splenocytes through flow cytometry on day 28. Data (n=10) are shown as mean±SEM and a one-way ANOVA with Tukey's multiple comparisons test was performed. The following comparisons were made with the dose 10 μg Ova and 1 mg SLA. **** SLA-1>AddaVax™-Ova; ***SLA-2>AddaVax™-Ova; ***SLA-3>AddaVax™-Ova. All samples were individually normalized to naive.

FIG. 9 shows a chromatogram obtained from chiral chromatography of optically inactive archaeol 3, displaying a highly complex chiral composition with more than 20 peaks for the same molecular ion [M+H]⁺ at m/z 653.65. This chromatogram was used to estimate the percentage of archaeol 3 corresponding to the single stereoisomer of archaeol 1. Since the latter elutes at a retention time of 144.2 minutes, the fraction of archaeol 3 corresponding to archaeol 1 was estimated by dividing the peak area at 144.2 minutes over the total area for all peaks found for archaeol 3.

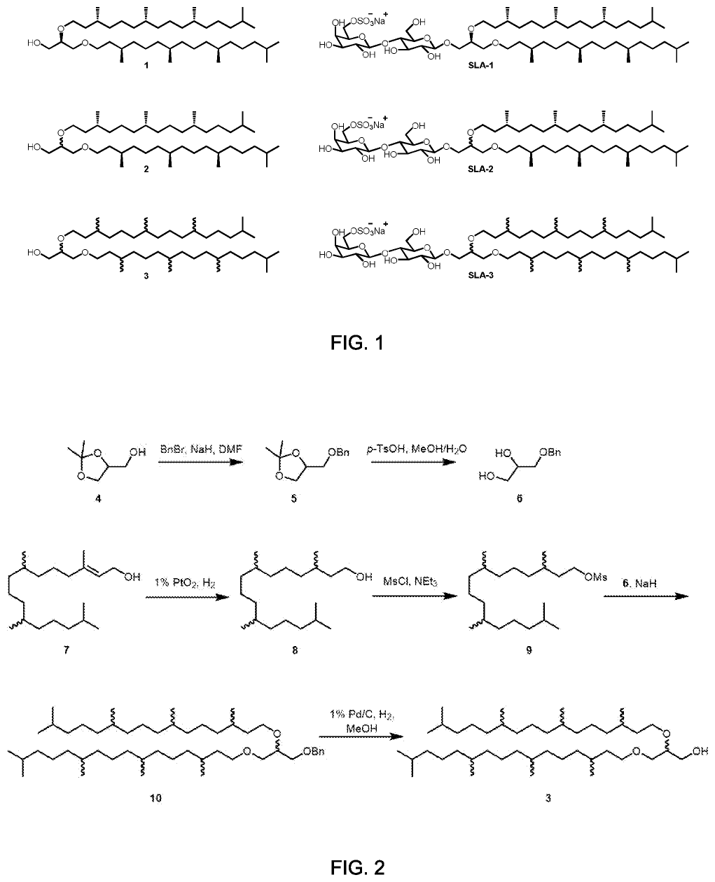

### DETAILED DESCRIPTION

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures, published sequences, and other references mentioned herein are expressly incorporated by reference in their entirety.

### Definitions

As used herein, the following terms may have meanings ascribed to them below, unless specified otherwise. However, it should be understood that other meanings that are known or understood by those having ordinary skill in the art are also possible, and within the scope of the present disclosure. In the case of conflict, the present specification, including definitions, will control. In addition, the materials, methods, and examples are illustrative only and not intended to be limiting.

The term “about” as used herein may be used to take into account experimental error, measurement error, and variations that would be expected by a person having ordinary skill in the art. For example, “about” may mean plus or minus 10%, or plus or minus 5%, of the indicated value to which reference is being made. “About” may also mean plus or minus the error margin of the measurement system employed to determine the value to which reference is being made.

As used herein the singular forms “a”, “an”, and “the” include plural references unless the context clearly dictates otherwise.

The phrase “and/or”, as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

As used herein, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used herein, the term “pharmaceutically acceptable carrier” refers to a carrier that is non-toxic. Suitable pharmaceutically acceptable carriers include, for example, one or more of water, saline, phosphate buffered saline, dextrose, glycerol, ethanol, and combinations thereof. Pharmaceutically acceptable carriers may further contain minor amounts of auxiliary substances such as wetting or emulsifying agents, preservatives or buffering agents that enhance shelf life or effectiveness.

As used herein, the term “pharmaceutically acceptable salt” refers to a derivative of the disclosed compound, wherein the parent compound is modified by making an acid or base salt thereof. Examples of pharmaceutically acceptable salts include, but are not limited to, mineral or organic acid salts of basic residues such as amines; alkali or organic salts of acidic residues such as carboxylic acids; and the like. Pharmaceutically acceptable salts include the conventional non-toxic salts or the quaternary ammonium salts of the parent compound formed, for example, from nontoxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

As use herein the term “adjuvant” refers to an agent that increases and/or directs specific immune responses to an antigen. Examples of adjuvants include, but are not limited to, adjuvants currently approved for used in human vaccines, including aluminum salts such as aluminum hydroxide, aluminum phosphate, and aluminum potassium sulfate (alum); CpG oligodeoxynucleotides (CpG ODN); oil-in-water emulsions (such as MF59 and AS03), AS04 (3′-O-deacylated monophosphoryl lipid A (MPL) plus aluminum salts), and AS01 (MPL and saponin QS-21 formulated in liposomes).

As used herein, the term “immunogenic composition” refers to a composition that is able to induce an immune response in a subject.

As used herein, the term “vaccine composition” refers to a composition comprising at least one antigen, or comprising a nucleic acid molecule encoding at least one antigen, in a pharmaceutically acceptable carrier, that is useful for inducing an immune response against the antigen in a subject, for the purpose of improving immunity against a disease and/or infection in the subject. Common examples of antigens include proteins, peptides, and polysaccharides. Some antigens include lipids and/or nucleic acids in combination with proteins, peptides and/or polysaccharides.

As used herein, the term “subject” refers to an animal, including both human and non-human animals. Examples of non-human subjects include, but are not limited to, pets, livestock, and animals used for antibody production and/or vaccine research and development. Examples of animals used for antibody production and/or vaccine research and development include, but are not limited to, rodents, rabbits, ferrets, non-human primates, swine, sheep, and cattle.

As used herein, the term “archaeol moiety” refers to a deprotonated “2,3-bis((3,7,11,15-tetramethylhexadecyl)oxy) propan-1-ol”. The archaeol moiety is the lipid portion of a sulfated lactosyl archaeol (SLA) glycolipid as described herein. An archaeol moiety comprises branched and fully saturated phytanyl chains attached at the sn-2,3-glycerol carbons via ether bonds. In an SLA glycolipid, the archaeol moiety is connected to the sugar moiety via an ether bond.

It should also be understood that, in certain methods described herein that include more than one step or act, the order of the steps or acts of the method is not necessarily limited to the order in which the steps or acts of the method are recited unless the context indicates otherwise.

### Details

The present inventors have developed a simple synthetic process for the production of optically inactive archaeol from optically inactive phytol. The resulting archaeol 3 comprises a mixture of stereoisomers, of which only about 6.5% were found to be in the 100% R configuration (i.e. having all seven chiral centers in the archaeol portion of the molecule in the R configuration). Surprisingly, the inventors found that SLA produced using this mixture of stereoisomers (SLA-3) was as effective an adjuvant as semi-synthetically produced SLA (SLA-1). The process developed by the present inventors may allow for simple, scalable, and more cost-effective production of sulfated glycolipid adjuvants, such as SLA, compared to existing processes.

The process comprises treating (±)-3-benzyloxy-1,2-propanediol with a mesylated phytol derivative through a double nucleophilic substitution reaction, followed by a reductive debenzylation reaction.

In an embodiment, the process comprises the following steps:

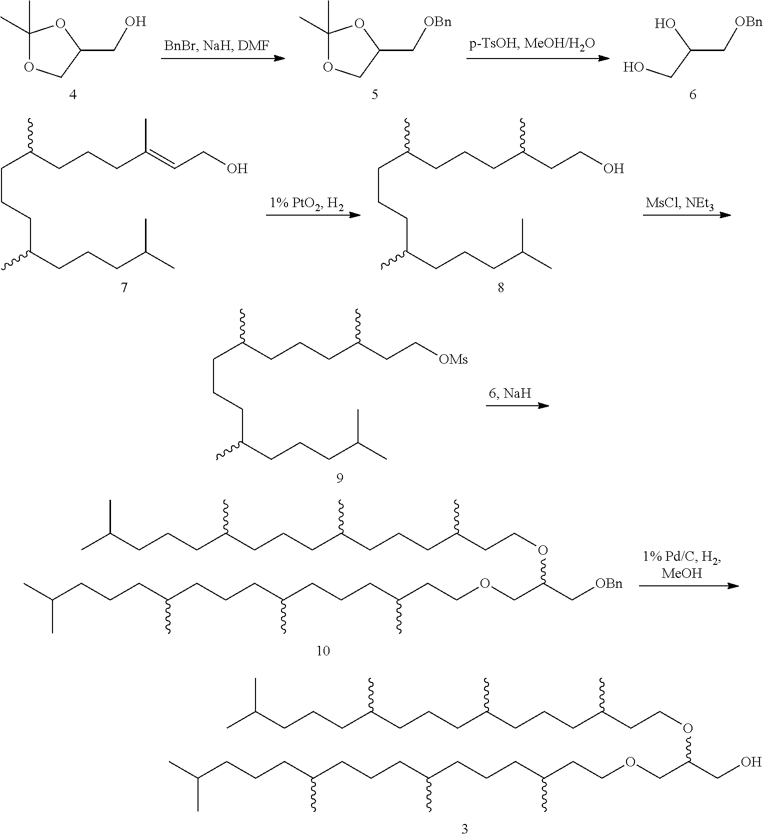

Further provided is process for synthesizing a mixture of two or more stereoisomers of a synthetic charged isoprenoid glycolipid or a pharmaceutically acceptable salt thereof, the process comprising covalently linking a sulfated saccharide group of the formula:

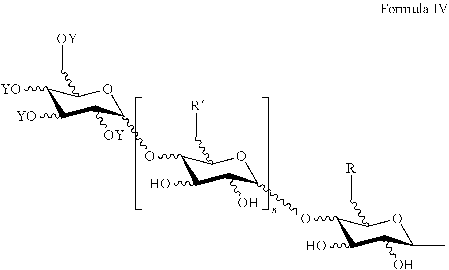

to the free sn-1 hydroxyl group of the glycerol backbone of an archaeol,
wherein the archaeol comprises a mixture of two or more stereoisomers of the structure:

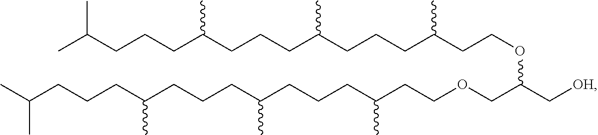

and wherein n is 0 or 1; R and R′ are independently hydrogen or hydroxyl; each Y is independently hydrogen or a sulfate group, and wherein at least one Y is a sulfate group; and less than 25%, less than 10%, about 5% to about 8%, or about 6.5% of the archaeol molecules in the mixture of two or more stereoisomers are of the configuration (R)-2,3-bis(((3R,7R,11R)-3,7,11,15-tetramethylhexadecyl)oxy) propan-1-ol. In an embodiment, the saccharide group is of the formula:

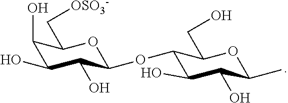

Further provided is a composition comprising a mixture of two or more stereoisomers of: a synthetic charged isoprenoid glycolipid comprising a sulfated saccharide group covalently linked to the free sn-1 hydroxyl group of the glycerol backbone of an archaeol moiety via a beta linkage, wherein the synthetic charged glycolipid is a compound of the formula:

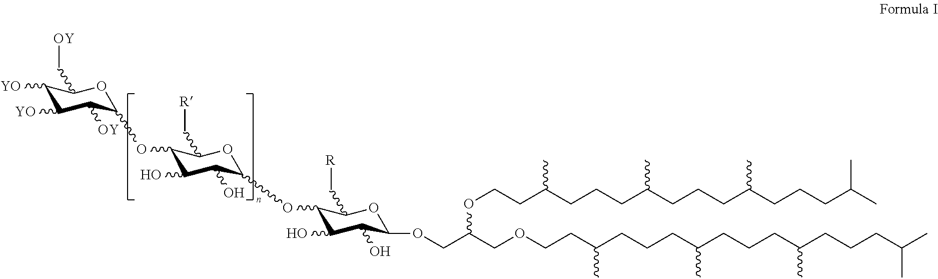

or a pharmaceutically acceptable salt thereof, wherein n is 0 or 1; R and R′ are independently hydrogen or hydroxyl; each Y is independently hydrogen or a sulfate group, and wherein at least one Y is a sulfate group; and less than 25%, less than 10%, about 5% to about 8%, or about 6.5% of the synthetic charged isoprenoid glycolipid molecules in the mixture comprise an archaeol moiety of the configuration (R)-2,3-bis(((3R,7R,11R)-3,7,11,15-tetramethylhexadecyl)oxy) propan-1-ol.

In an embodiment, only one Y is a sulfate group. In an embodiment, n is 0 and R is OH.

The composition may comprise two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more stereoisomers of the synthetic charged isoprenoid glycolipid. In an embodiment, the composition comprises 15 to 128 stereoisomers of the synthetic charged isoprenoid glycolipid.

In an embodiment, the sulfated saccharide group comprises monosaccharide moieties selected from the group consisting of mannose (Man), glucose (Glc), rhamnose (Rha) and galactose (Gal) moieties. In a further embodiment, the compound comprises a sulfate group at the 6′ position of the terminal monosaccharide moiety.

In an embodiment, the compound is 6′-sulfate-α-D-Manₚ-(1,6)-β-D-Galₚ-(1,4)-β-D-Glcₚ-(1,1)-archaeol, or 6′-sulfate-β-D-Glcₚ-(1,6)-β-D-Galₚ-(1,4)-β-D-Glcₚ-(1,1)-archaeol, or 6′-sulfate-β-D-Galₚ-(1,4)-β-D-Glcₚ-(1,6)-β-D-Glcₚ-(1,1)-archaeol.

In an embodiment, the sulfated saccharide group is a sulfated lactosyl group. In an embodiment, the sulfated lactosyl group is a 6′-S-lactosyl group. In an embodiment, the 6′-S-lactosyl group is 6′-sulfate-β-D-Galₚ-(1,4)-β-D-Glcₚ.

In an embodiment, the synthetic charged glycolipid is a compound of the structure:

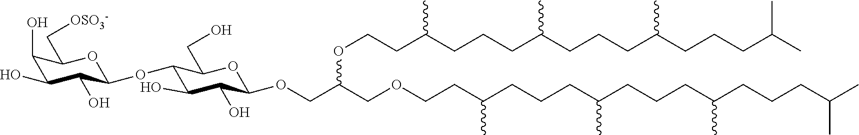

or a pharmaceutically acceptable salt thereof.

Further provided is an archaeosome comprising a synthetic charged glycolipid composition as described herein. The archaeosome or synthetic charged glycolipid composition as described herein may be used as an adjuvant to enhance or direct an immune response in a subject. The subject may be a human or non-human animal, such as, but not limited to, a companion animal or livestock animal. The archaeosome may further be used as an adjuvant in a vaccine or immunogenic composition and/or for the manufacture of a vaccine or immunogenic composition.

The synthetic charged glycolipid composition or archaeosome may be included in an immunogenic composition together with an antigen, such as but not limited to a peptide, protein, or virus-like particle. The immunogenic composition may be a vaccine composition. The immunogenic composition may further comprise a pharmaceutically acceptable carrier and/or an additional adjuvant other than a synthetic charged isoprenoid glycolipid. Examples of suitable additional adjuvants include but are not limited to poly(I:C), CpG ODN, Pam₃CSK₄, MPLA, R848, and saponins. Poly(I:C) and CpG ODN may be of particular interest, as semi-synthetic SLA has been shown to have strong synergy with these adjuvants.¹⁴

Immunogenic compositions as described herein may be used to induce an immune response in a subject. The subject may be a human or non-human animal, such as but not limited to a companion animal or livestock animal.

### EXAMPLES

The following non-limiting examples are illustrative of the present disclosure.

### Example 1: Synthesis of Sulfated Lactosyl Archaeols (SLAs)

### Materials and Methods

Unless stated otherwise, all reactions were performed under an argon atmosphere. All commercially available solvents and reagents used were purchased from Sigma Aldrich, unless indicated otherwise and were used without further purification. The phytol 7 was optically inactive and consisted of a 97% mixture of isomers. The biological archaeol 1 was prepared by the inventors while the synthetic archaeol 2 was procured from Avanti. Reactions were monitored by TLC analysis using UV254 pre-coated TLC plates and visualized under UV light or by staining with potassium permanganate (KMnO₄) or sulfuric acid (H₂SO₄) in methanol. ¹H and ¹³C NMR spectra were obtained in the specified deuterated solvents using a Bruker AVANCE III 500 MHz spectrometer equipped with a TXI 5 mm room-temperature probe or a Bruker AVANCE III 700 MHz spectrometer equipped with a 5 mm TCI cryoprobe, as indicated. The NMR data are presented as follows: chemical shift δ (ppm), multiplicity, coupling constant and integration. The following abbreviations were used to explain the multiplicities: s=singlet, d=doublet, t=triplet, m=multiplet, bs=broad singlet. The ¹H NMR and ¹³C NMR spectra were referenced using 3-(trimethylsilyl)-1-propanesulfonic acid sodium salt (TMS=0.0) as the internal reference.

Synthesis of compound 5 (FIG. 2). In a 500 mL 3-neck round bottom flask containing DMF (210 mL), sodium hydride (2.27 g, 56.8 mmol) was added under stirring. The resulting suspension was cooled to −4° C., followed by the dropwise addition of isopropylidene glycerol (4) (4.7 mL, 37.8 mmol) over 30 min. The resulting mixture was stirred at −4° C. for 30 min, then allowed to warm up to room temperature. Benzyl bromide (5.4 mL, 45.5 mmol) was added dropwise over 60 min and stirred at room temperature for 1.5 h. Upon completion of reaction as monitored by TLC, the reaction mixture was cooled to 0° C. and quenched by the slow addition of a saturated solution of ammonium chloride until gas evolution was no longer visible. Dichloromethane was added and the layers were separated. The aqueous layer was re-extracted with dichloromethane. The combined organic layers were washed with water and brine, dried over Na₂SO₄, filtered, and concentrated. The crude oil was purified by flash chromatography (5-10% EtOAc in hexanes) to afford compound 5 (7.86 g, 93%) as a colorless oil. [α]_{D}²³=0.00° (c=1.45, CHCl₃); ¹HNMR (CDCl₃, 500 MHz) δ 7.38-7.29 (m, 5H), 4.59 (m, 2H), 4.31 (m, 1H), 4.07 (dd, 1H, J1=8.3 Hz, J2=6.5 Hz, 3.76 (dd, 1H, J1=8.3 Hz, J2=6.4 Hz), 3.57 (dd, 1H, J1=9.8 Hz, J2=5.7 Hz), 3.49 (dd, 1H, J1=9.8 Hz, J2=5.6 Hz), 1.43 (s, 3H), 1.38 (s, 3H); ¹³CNMR (CDCl₃, 176 MHz) δ 138.1 128.6, 127.9, 127.9, 109.6, 74.9, 73.7, 71.2, 67.0, 26.9, 25.5 ppm; HRMS (ESI+) calcd. for C₁₃H₁₈O₃Na [M+Na]⁺: 245.1148, found 245.1150.

Synthesis of compound 6 (FIG. 2). Para-toluenesulfonic acid monohydrate (3.6 g, 18.9 mmol) was added to a stirred solution of compound 5 (8.42 g, 37.9 mmol) in methanol (62 mL) and water (26 mL) in a 250 mL round bottom flask. The resulting solution was stirred at room temperature for 16 h. Once end of reaction was confirmed by TLC, the reaction mixture was neutralized with 10% NaHCO₃ until pH=6. Ethyl acetate was added and the layers were separated. The aqueous layer was re-extracted with ethyl acetate. The combined organic layers were washed with brine and dried over Na₂SO₄, filtered, and concentrated to afford 3-(benzyloxy)propane-1,2-diol 6 (5.1 g, 71% yield) as an off-white solid. No further purification was required and the product was used as is for the next step. [α]_{D}²⁴=0.00° (c=1.28, CHCl₃); ¹HNMR (CDCl₃, 500 MHz) δ 7.39-7.30 (m, 5H), 4.57 (s, 2H), 3.93-3.89 (m, 1H), 3.73 (dd, 1H, J1=11.4 Hz, J2=3.9 Hz), 3.65 (dd, 1H, J1=11.4 Hz, J2=5.4 Hz), 3.62-3.55 (m, 2H), 2.63 (br s, 1H), 2.12 (br s, 1H); ¹³CNMR (CDCl₃, 176 MHz) δ137.8, 128.7, 128.1, 127.9, 73.7, 72.0, 70.7, 64.2 ppm; HRMS (ESI+) calcd. for C₁₀H₁₄O₃Na [M+Na]⁺: 205.0835, found 205.0839.

Synthesis of compound 8 (FIG. 2). In a 500 mL round bottom flask equipped with a magnetic stirrer, phytol (7) (20.0 g, 67.5 mmol) was stirred in tetrahydrofuran (140 mL). Nitrogen was sparged in the resulting solution for 10 min, followed by addition of platinum oxide (153 mg, 0.67 mmol). The resulting suspension was purged three times with cycles of nitrogen and hydrogen gas, and a septum was put on the reaction flask. A balloon of hydrogen was inserted through the septum, and the reaction mixture was stirred at room temperature overnight. Once the reaction completion was confirmed by NMR, the reaction mixture was filtered on a bed of Celite and the cake was washed with tetrahydrofuran. The filtrate was concentrated under reduced pressure and the residue was purified by flash chromatography (5% EtOAc in hexanes) to afford compound 8 (20.1 g, 91%) as a colorless oil. [α]_{D}²³=0.000 (c=1.41, CHCl₃); ¹HNMR (CDCl₃, 500 MHz) δ 3.74-3.65 (m, 2H), 1.65-1.49 (m, 4H), 1.39-1.04 (m, 21H), 0.91-0.85 (m, 15H); ¹³CNMR (CDCl₃, 176 MHz) δ 61.4, 40.2, 40.1, 39.5, 37.6, 37.6, 37.5, 37.5, 37.4, 32.9, 32.9, 29.7, 28.1, 25.0, 24.6, 24.5, 22.9, 22.8, 19.9, 19.9, 19.8 ppm; HRMS (ESI+) calcd. for C₂₀H₄₂ONa [M+Na]⁺: 321.3128, found 321.3119.

Synthesis of compound 9 (FIG. 2). Triethylamine (5.1 mL, 36.8 mmol) was added to a stirred solution of dihydrophytol (8) (10.0 g, 33.5 mmol) in dichloromethane (50 mL) in a 250 mL round bottom flask. The resulting solution was cooled to 0° C. and methanesulfonyl chloride (3.1 mL, 40.2 mmol) was added dropwise. The resulting white suspension was stirred at 0° C. for 1 h, and allowed to warm up to room temperature and stirred overnight. Once the reaction completion was confirmed by NMR, the reaction was quenched by slowly adding water. The layers were separated, and the aqueous layer was neutralized with 10% NaHCO₃ solution and re-extracted with dichloromethane. The combined organic layers were washed with brine, dried over MgSO₄, filtered, and concentrated under reduced pressure to afford compound 9 (12.25 g, 97% yield) as a pale yellow oil. The crude product was used for next step without further purification. This compound was prone to decomposition. [α]_{D}²³=0.00° (c=0.77; 1:1 CH₂Cl₂/MeOH); ¹HNMR (CDCl₃, 500 MHz) δ 4.32-4.24 (m, 2H), 3.01 (s, 3H), 1.82-1.77 (m, 1H), 1.63-1.49 (m, 2H), 1.39-1.09 (m, 21H), 0.93 (d, J=6.5 Hz, 3H), 0.88-0.85 (m, 12H).

Synthesis of compound 10 (FIG. 2). In a 100 mL round bottom flask equipped with a magnetic stirrer, compound 6 (0.25 g, 1.37 mmol) was stirred in N,N-dimethylformamide (7.5 mL). The resulting solution was cooled to 0° C. and sodium hydride (0.14 g, 3.43 mmol) was added portion-wise. The resulting suspension was stirred at 0° C. for 20 min, and then allowed to warm up to room temperature where a solution of compound 9 (1.29 g, 3.43 mmol) in N,N′-dimethylformamide (12 mL) was added dropwise over 1 h using an addition funnel. The reaction mixture was heated to 60° C. and stirred at this temperature for 2 h. The reaction mixture was then cooled down to room temperature and was quenched with water. Ethyl acetate was added and the layers were separated. The aqueous layer was re-extracted with ethyl acetate. The combined organic layers were washed with water and brine, dried over MgSO₄, filtered, and concentrated under reduced pressure to afford a yellow oil. The residue was purified by flash chromatography (1% EtOAc in hexanes) to afford product 10 (0.54 g, 53% yield) as a colorless oil. [α]_{D}²³=0.00° (c=0.60; 1:1 CH₂Cl₂/MeOH); ¹HNMR (CDCl₃, 500 MHz) δ 7.34-7.26 (m, 5H), 4.55 (s, 2H), 3.63-3.42 (m, 9H), 1.61-1.50 (m, 4H), 1.36-1.06 (m, 44H), 0.87-0.84 (m, 30H); ¹³CNMR (CDCl₃, 176 MHz) δ 138.6, 128.5, 127.7, 127.7, 79.1, 73.5, 71.0, 70.9, 70.4, 70.1, 70.1, 69.1, 69.0, 39.5, 37.7, 37.6, 37.6, 37.5, 37.5, 37.4, 37.3, 37.2, 36.9, 36.8, 33.0, 32.9, 32.9, 30.0, 30.0, 30.0, 29.9, 28.1, 25.0, 25.0, 24.7, 24.6, 24.5, 22.9, 22.8, 19.9, 19.9, 19.8, 19.8, 19.8 ppm; HRMS (ESI+) calcd. for C₅₀H₉₄O₃Na [M+Na]⁺: 765.7095, found 765.7107.

Synthesis of compound 3 (FIG. 2). In a 500 mL round bottom flask containing a stirred solution of compound 10 (0.54 g, 0.70 mmol) in tetrahydrofuran (10 mL), nitrogen was sparged for 10 min and palladium on carbon (0.070 g, 0.07 mmol) was added. The resulting suspension was purged three times with cycles of nitrogen and hydrogen gas and a septum was put on the reaction flask. A balloon of hydrogen was inserted through the septum and the reaction mixture was stirred at room temperature overnight. Once the reaction completion was confirmed by TLC, the reaction mixture was filtered on a bed of Celite and the cake was washed with tetrahydrofuran. The filtrate was combined with washings and concentrated under reduced pressure. The residue was purified using flash chromatography (1-5% EtOAc in hexanes) to afford a mixture of stereoisomers of archaeol 3 (0.34 g, 72% yield) as a colorless oil. [α]_{D}²⁴=0.00° (c=1.30, CHCl₃); ¹HNMR (CDCl₃, 500 MHz) δ 3.75-3.46 (m, 9H), 1.63-1.51 (m, 4H), 1.39-1.07 (m, 43H), 0.90-0.85 (m, 30H); ¹³CNMR (CDCl₃, 176 MHz) δ 78.4, 78.4, 78.4, 71.2, 71.1, 71.1, 71.1, 70.3, 68.8, 68.8, 63.3, 63.3, 39.5, 37.6, 37.6, 37.6, 37.6, 37.6, 37.5, 37.5, 37.5, 37.5, 37.4, 37.3, 37.3, 32.9, 32.9, 30.0, 30.0, 30.0, 28.1, 25.0, 25.0, 24.6, 24.6, 24.5, 22.9, 22.8, 19.9, 19.9, 19.9, 19.8, 19.8, 19.8 ppm; HRMS (ESI+) calcd. for C₄₃H₈O₃Na [M+Na]⁺: 675.6626, found 675.6634.

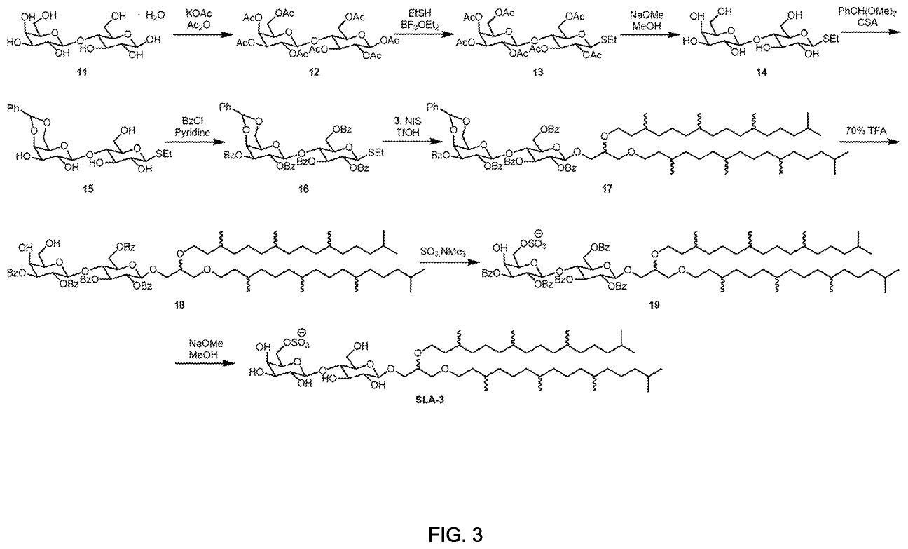

Synthesis of compound 17 (FIG. 3). In a 500 mL round bottom flask, donor 16^{16, 18} (4.6 g, 4.6 mmol) and archaeol 3 (2.50 g, 3.8 mmol) were dissolved in dichloromethane under argon, then dried for 24 h under high vacuum. Activated powdered 4 Å molecular sieves (about 10 g) were dried in an oven at 105° C. for 48 h. Prior to the reaction, a stir bar and the dried molecular sieves were flame-dried thoroughly in a round bottom flask, along with a funnel. The flame-dried molecular sieves and stir bar were added to the round bottom flask containing the vacuum dried starting materials (donor 16 and archaeol 3). Anhydrous dichloromethane (200 mL) was used to dissolve the starting materials and the mixture was stirred under argon at room temperature for 15 min. The reaction mixture was then cooled to −50° C. and stirred for another 15 min. N-iodosuccinimide (2.16 g, 11.5 mmol) and trifluoromethanesulfonic acid (0.52 mL, 5.9 mmol) were quickly added to the reaction and stirring was maintained. Within 1 h, a deep red colour was observed and the temperature was allowed to warm up to −20° C. The reaction was monitored by TLC using 7:2:1 hexanes/ethyl acetate/dichloromethane. The reaction mixture was diluted with dichloromethane, filtered by vacuum filtration, and rinsed with additional dichloromethane. The resulting filtrate was washed with 10% sodium thiosulfate (500 mL), a saturated sodium bicarbonate solution (500 mL) and brine (500 mL). The organic layer was dried over MgSO₄, filtered by vacuum filtration, and evaporated to dryness. The residue was purified by flash chromatography by eluting with 8:1:1, 7:1:1 and 7:2:1 hexanes/ethyl acetate/dichloromethane to yield compound 17 as a viscous colorless oil (3.83 g, 63% yield). [α]_{D}²³=67.65° (c=0.68, 1:1 CH₂Cl₂/MeOH); ¹HNMR (CDCl₃, 500 MHz) δ 8.01-7.87 (m, 10H), 7.58-7.15 (m, 20H), 5.85-5.77 (m, 2H), 5.35-5.28 (m, 1H), 5.16-5.14 (m, 1H), 4.84-4.83 (m, 1H), 4.77-4.74 (m, 1H), 4.62-4.60 (m, 1H), 4.38-4.36 (m, 1H), 4.30-4.29 (m, 1H), 4.22 (t, 1H, J=9.0 Hz), 3.84-3.76 (m, 2H), 3.58-3.09 (m, 10H), 2.95 (s, 1H), 1.54-1.03 (m, 49H), 0.87-0.67 (m, 30H); ¹³CNMR (CDCl₃, 176 MHz) δ 166.3, 165.8, 165.4, 165.2, 165.0, 137.6, 133.5, 133.3, 133.1, 130.0, 130.0, 129.8, 129.8, 129.7, 129.5, 129.0, 128.9, 128.6, 128.5, 128.4, 128.4, 128.1, 126.5, 101.6, 101.1, 100.7, 74.1, 73.2, 72.9, 72.7, 72.4, 70.6, 70.1, 70.0, 69.5, 69.0, 68.1, 66.6, 62.5, 39.5, 37.6, 37.5, 37.4, 37.1, 36.7, 32.9, 29.9, 29.9, 28.1, 25.0, 24.6, 24.5, 22.9, 22.8, 19.9, 19.8, 19.8, 19.7, 19.6 ppm; HRMS (ESI+) calcd. for C₉₇H₁₃₂O₁₈Na [M+Na]⁺: 1607.9306, found 1607.9288.

Synthesis of compound 18 (FIG. 3). Benzylidene 17 (3.80 g, 2.40 mmol) was dissolved in dichloromethane (290 mL) and cooled with an ice bath while stirring. 70% aqueous trifluoroacetic acid (TFA, 33 mL) was added and the reaction was monitored by TLC. After 3.5 h, TLC still showed unreacted starting material. Neat TFA (15 mL) was then added dropwise and the reaction was stirred for an additional 30 min. The reaction was then diluted with water and transferred to a separatory funnel with further rinsing by dichloromethane and water. The organic phase was washed with saturated sodium bicarbonate solution and brine, and dried with sodium sulfate. Gravity filtration followed by evaporation to dryness gave the crude product, which was purified by flash chromatography. Elution was performed with hexanes/ethyl acetate/dichloromethane (7:2:1, 6:3:1, 5:3.5:1.5 and 4:4:2) to yield compound 18 as a waxy white solid (3.13 g, 87% yield). [α]_{D}²³=47.22° (c=0.72, 1:1 CH₂Cl₂/MeOH); ¹HNMR (CDCl₃, 500 MHz) δ 8.04-7.90 (m, 10H), 7.60-7.55 (m, 2H), 7.51-7.41 (m, 6H), 7.38-7.32 (m, 5H), 7.25 (m, 2H), 5.75-5.72 (m, 2H), 5.44-5.40 (m, 1H), 5.07-5.05 (m, 1H), 4.77-4.74 (m, 2H), 4.6 (m, 1H), 4.43-4.41 (m, 1H), 4.2-4.17 (m, 2H), 3.84-3.82 (m, 2H), 3.43-3.13 (m, 12H), 1.53-1.02 (m, 48H), 0.87-0.68 (m, 30H); ¹³CNMR (CDCl₃, 176 MHz) δ 166.0, 165.9, 165.5, 165.3, 165.2, 133.6, 133.5, 133.4, 133.4, 130.0, 130.0, 129.9, 129.8, 129.7, 129.4, 129.4, 129.1, 128.9, 128.8, 128.6, 128.5, 128.5, 125.0, 101.4, 101.2, 101.2, 76.5, 74.4, 74.2, 73.7, 73.6, 73.0, 73.0, 72.0, 71.9, 70.4, 70.1, 70.0, 69.9, 69.7, 69.3, 69.0, 68.4, 62.7, 39.5, 37.7, 37.6, 37.6, 37.5, 37.4, 37.2, 37.1, 37.0, 36.7, 36.6, 32.9, 30.0, 30.0, 29.9, 29.9, 29.8, 29.8, 28.1, 25.0, 25.0, 24.6, 24.5, 24.5, 22.9, 22.8, 19.9, 19.8, 19.8, 19.7, 19.7, 19.7, 19.6, 19.6, 19.6, 19.5, 19.4 ppm; HRMS (ESI+) calcd, for C₉₀H₁₂₈O₁₈Na [M+Na]⁺: 1519.8993, found 1519.8993.

Synthesis of compound 19 (FIG. 3). Diol 18 (3.13 g, 2.10 mmol) was dissolved in anhydrous dichloromethane (28 mL) and pyridine (14 mL) with stirring under argon at room temperature. Trimethylamine sulfur trioxide complex (1.15 g, 8.30 mmol) was added in one portion. The reaction was monitored by TLC, which did not show the presence of diol 18 after 1.75 h, and the emergence of a more polar spot on the TLC. The reaction mixture was evaporated and the residue was purified by flash chromatography by eluting with 10:90:0.2 methanol/dichloromethane/pyridine to yield compound 19 (3.13 g, 950% yield) as a white solid. [α]_{D}²³=40.00° (c=0.70, CHCl₃); ¹HNMR (1:1 CD₃OD/CD₂Cl₂, 700 MHz) δ 8.02-7.87 (m, 10H), 7.62-7.60 (m, 1H), 7.57-7.52 (m, 2H), 7.50-7.45 (m, 5H), 7.41-7.38 (m, 2H), 7.34-7.30 (m, 3H), 7.24-7.22 (m, 2H), 5.72 (td, 1H, J1=4.9 Hz, J2=9.1 Hz), 5.62 (dd, 1H, J1=7.7 Hz, J2=10.5 Hz), 5.33-5.29 (m, 1H), 5.16 (dd, 1H, J1=3.5 Hz, J2=10.5 Hz), 4.87 (dd, 1H, J1=2.1 Hz, J2=6.3 Hz), 4.81-4.78 (m, 1H), 4.47-4.44 (m, 1H), 4.25 (td, 1H, J1=2.8 Hz, J2=9.8 Hz, 1H), 4.16 (d, 1H, J=2.8 Hz), 3.87-3.13 (m, 13H), 1.52-0.69 (m, 84H); ¹³CNMR (1:1 CD₃OD/CD₂Cl₂, 176 MHz) δ 166.9, 166.7, 166.6, 166.3, 166.1, 134.3, 134.0, 134.0, 133.9, 133.9, 130.4, 130.3, 130.3, 130.3, 130.2, 130.1, 130.1, 129.7, 129.3, 129.2, 129.1, 129.1, 129.0, 125.7, 102.1, 102.1, 102.1, 101.8, 101.7, 78.4, 78.3, 78.2, 78.1, 77.2, 74.8, 74.1, 74.0, 73.8, 73.8, 73.6, 73.0, 73.0, 71.0, 70.5, 69.7, 69.4, 66.2, 64.5, 63.3, 45.5, 40.1, 38.1, 38.0, 38.0, 38.0, 37.7, 37.6, 37.6, 37.2, 37.2, 33.5, 33.5, 30.5, 30.4, 30.3, 30.3, 28.7, 25.5, 25.5, 25.1, 25.0, 23.0, 22.9, 20.1, 20.1, 20.0, 20.0, 20.0, 20.0, 19.9, 19.9, 19.8, 19.8, 19.7 ppm; HRMS (ESI−) calcd, for C₉₀H₁₂₈O₂₁S [M−H]⁻: 1575.8596, found 1575.8571.

Synthesis of SLA-3 (FIG. 3). Protected sulfated glycolipid 19 (3.50 g, 2.20 mmol) was dissolved in dry dichloromethane (65 mL) under argon. Methanolic sodium methoxide (0.5 M in methanol, 63 mL) was added, and the mixture was stirred for 15 min at room temperature. The reaction mixture was then refluxed at 45° C. for 3 h or until no more starting material was present on the TLC. Once the reaction was completed, it was allowed to cool to room temperature. Methanol, chloroform and sodium acetate buffer (sodium acetate and acetic acid, pH 5) were added and the mixture was stirred for 16 h. The mixture was then transferred into a separatory funnel, followed by the additions of chloroform and buffer. The bottom organic layer was separated and the aqueous phase was washed with chloroform. The combined organic layers were dried with sodium sulfate, gravity filtered, and concentrated by evaporation. The residue was dissolved in chloroform and transferred to a separatory funnel and further washed with saturated sodium bicarbonate solution. The organic layer was dried with Na₂SO₄, filtered by gravity and concentrated to dryness. The residue was purified by flash chromatography by eluting with 1-10% methanol/dichloromethane to yield SLA-3 (1.5 g, 64% yield) as a white solid. [α]_{D}²³=−1.19° (c=0.84, 1:1 CH₂Cl₂/MeOH); ¹HNMR (1:1 CD₃OD/CD₂Cl₂, 700 MHz) δ 4.32-4.26 (m, 3H), 4.11-4.09 (m, 1H), 3.94-3.93 (m, 1H), 3.87-3.81 (m, 4H), 3.66-3.46 (m, 12H), 3.42-3.41 (m, 1H), 1.61-1.49 (m, 6H), 1.38-1.08 (m, 45H), 0.89-0.84 (m, 30H); ¹³CNMR (1:1 CD₃OD/CD₂Cl₂, 176 MHz) δ 105.0, 103.6, 83.0, 78.7, 75.6, 75.2, 75.2, 74.4, 74.1, 73.8, 71.7, 70.9, 70.8, 69.8, 69.2, 67.6, 62.1, 40.0, 38.1, 38.1, 38.0, 37.9, 33.5, 30.6, 28.7, 25.5, 25.1, 25.1, 25.1, 22.9, 22.8, 20.1, 20.0 ppm; HRMS (ESI−) calcd. for C₅₅H₁₁₈O₁₆S [M−H]⁻: 1055.7285, found 1055.7251.

Preparation of Archaeol (1) from Halobacterium salinarum:

Halobacterium salinarum (ATCC 33170) was grown under aerobic conditions at 37° C. in the following medium: 15.0 g/L bacteriological peptone, 2.24 g/L KCl, 2.94 g/L sodium citrate and 19.72 g/L MgSO₄·7H₂O. After 47 h of incubation, the biomass was harvested and extracted for lipids in a mixture of chloroform/methanol/water followed by precipitation of total polar lipids (TPL) using cold acetone. Methanolic hydrolysis of the TPL was done in a mixture of acetyl chloride/methanol under reflux at 63° C. for 4 h. An archaeol-rich fraction was partitioned into petroleum ether from a two-phase solvent system made of petroleum ether/methanol/water. The archaeol-rich fraction was applied to a silica gel 60 column using a step-gradient program of hexane and methyl tert-butyl ester (MTBE). Pure archaeol fractions were combined and characterized using TLC, mass spectrometry, NMR and optical rotation. A typical yield of pure archaeol 1 is 1% (w/w) of cell biomass dry weight.

### Archaeol and SLA Nomenclature:

Three SLA samples (SLA-1, SLA-2 and SLA-3) were synthesized using three different sources of archaeol. SLA-1 is a semi-synthetic compound, produced using archaea-derived archaeol 1 of 100% R stereoisomer from Halobacterium salinarum, according to a previously reported procedure,¹⁷ with slight modifications as described herein. The two fully synthetic SLA samples, SLA-2 and SLA-3, were prepared from synthetic archaeols. SLA-2 was prepared using archaeol 2 purchased from Avanti, consisting of epimers—94% (R)-2,3-bis(((3R,7R,11R)-3,7,11,15-tetramethylhexadecyl)oxy) propan-1-ol and 6% (S)-2,3-bis(((3R,7R,11R)-3,7,11,15-tetramethylhexadecyl)oxy) propan-1-ol). SLA-3 (FIG. 3) was synthesized from the mixture of stereoisomers of archaeol 3 (2,3-bis((3,7,11,15-tetramethylhexadecyl)oxy) propan-1-ol, FIG. 2).

### Results and Discussion

To evaluate the impact of SLA's stereochemistry on the adjuvanticity of the potential vaccine adjuvant towards Ova, three different sources of archaeol were used to prepare the corresponding SLAs. The convergent total synthesis of the archaeol, 2,3-bis((3,7,11,15-tetramethylhexadecyl)oxy) propan-1-ol (3), is outlined in FIG. 2. Archaeol has seven stereocenters. In nature, it is derived from phytol where the chiral centers at C-7 and C-11 positions adopt the R configuration exclusively. Phytanic acid, on the other hand, may adopt the R or S configuration at the C-3 position depending on the organisms it originates from and hence results into diastereomers.²⁰ The microbial archaeol, (R)-2,3-bis(((3R,7R,11R)-3,7,11,15-tetramethylhexadecyl)oxy) propan-1-ol (1), exists as a single stereoisomer of 100% R configuration and was isolated from Halobacterium salinarum, as described herein. The commercially available archaeol purchased from Avanti (2) consisted of two diastereomers, with different spatial arrangements of the ether oxygen at the C-2 position—about 94% of (R)-2,3-bis(((3R,7R,11R)-3,7,11,15-tetramethylhexadecyl)oxy) propan-1-ol and 6% of (S)-2,3-bis(((3R,7R,11R)-3,7,11,15-tetramethylhexadecyl)oxy) propan-1-ol. The stereoisomeric mixture of archaeol 3 was synthesized from commercially available starting materials, racemic isopropylidene glycerol 4 and optically inactive phytol 7 through a non-stereoselective synthetic route. The phytol used for the synthesis was a mixture of diastereomers and was optically inactive, and given that archaeol has 7 chiral centers, one could expect up to 128 configurations. A benzylation reaction on the hydroxyl group of compound 4, followed by acid hydrolysis of the compound 5, produced (±)-3-benzyloxy-1,2-propanediol 6 in good yield. The diol 6 is also a commercially available material and can be used directly to couple with the mesylate 9 to reduce the number of synthetic steps. In parallel, the commercially available phytol 7 was reduced using catalytic hydrogenation to afford saturated alcohol 8, which was then converted to the compound 9. The latter was found to be unstable at ambient temperature and was immediately utilized in a sodium hydride-assisted dialkylation reaction of diol 6 without further purification to afford the protected archaeol 10 in moderate yield. Finally, removal of the benzyl functional group through catalytic hydrogenation of 10 afforded archaeol 3 as a mixture of stereoisomers.

Three SLA samples were synthesized. With the desired undefined archaeol 3 in hand, procedures already described in the literature^{16, 18} were utilized to produce a fully synthetic SLA (SLA-3). FIG. 3 illustrates the synthetic path used to produce SLA-3, starting from lactose hydrate 11. The latter was subjected to a series of protection, nucleophilic substitution and deprotection steps to produce the protected thioglycoside 16. The glycosylation step between the thioglycoside 16 and the archaeol 3 was adapted from Fraser-Reid and co-workers²¹ where a combination of N-iodosuccinimide and triflic acid was employed for easier manipulation, and the protected glycolipid 17 was obtained in a comparable yield. Subsequent removal of the benzylidene in acidic conditions, followed by sulphonation and deprotection of the benzoyl groups in dilute basic medium, afforded SLA-3. Another fully synthetic SLA (SLA-2) was prepared using commercially available C-2 enantioenriched archaeol 2 (94% R-form). Finally, a sample of the semi-synthetic SLA (SLA-1) was synthesized and characterized using archaeol (1) derived from Halobacterium salinarum.

### Example 2: Characterization of Archaeols and SLAs

### Materials and Methods

### Optical Rotation:

The optical rotations of the archaeols were measured on an automatic Rudolph Autopol I polarimeter (Table 1) and the values are reported in g/100 mL concentration using a 100 mm polarimeter cell. Control experiments were also carried to verify the validity of the measurements (Table 1S, SI).

### LC-HRMS Analysis:

Each archaeol was dissolved at 10 μg/mL in methanol and the resulting solution was analyzed using a Bruker MicrOTOF-Q mass analyzer attached to an HPLC system (Agilent 1200 Series) equipped with a DA detector. The samples were analyzed by infusion, in which the sample is infused into the mobile phase flow and passes directly into the mass spectrometer and by injection (2 μL) using a C8 column (Halo 3.0 mm ID×50 mm, 2.7 μm, advance material technology) at 40° C. The mobile phase consisted of 5 mM ammonium acetate and methanol at a flow rate of 0.5 mL min⁻¹. A gradient of methanol from 95% to 100% within 2 min was used to elute the compound. UV detection was scanning across 190-950 nm. For mass analysis, positive electrospray ionization mode (ES+) was used. Mass range was selected from 100 to 1500 Da. The MS was operated in full scan and auto-MS/MS modes using Nitrogen for CID (collision-induced dissociation) to form product ions. Mass was calibrated using ESI-Low concentration Tuning Mix (Agilent). Presumed chemical formulae, error (ppm) and mSigma were calculated using the SmartFormula calculator (Bruker).

### LC-MS Analysis:

Each archaeol was dissolved at 10 μg/mL in methanol and the resulting solution was analyzed using a Shimadzu LC-MS2020 mass analyzer with an HPLC system (Prominence). The samples were analyzed by injection (2 μL) using a chiral column (Lux i-Amylose 3 4.6 mm ID×250 mm, 3.0 μm, Phenomenex Inc.) at 30° C. The mobile phase consisted of methanol:H₂O 95:5% v/v at a flow rate of 0.4 mL min⁻¹. A post-column addition of 5 mM ammonium acetate at a flow of 0.1 mL min⁻¹ was added to promote ionization and also create an ammonium adduct. For mass analysis, positive electrospray ionization mode (ES+) was used. The MS was operated in SIM (Selected Ion Monitoring) mode. Mass was calibrated using LC-MS2020 Tuning Mix (Shimadzu).

### Results and Discussion

Many biologically active molecules have chiral centers and since the human body differentiates between enantiomers (or stereoisomers), it is important to know the structural impact a molecule can have upon administration. Despite these impacts, a large number of drugs are marketed as racemates where one enantiomer provokes the active response, while the other enantiomer is either inactive or has a lower activity. This often arises from economic considerations—a racemate is significantly cheaper to produce than producing a single stereoisomer or separating one from the other one. Since the sugar moiety of the SLAs is already endowed with a built-in chiral fragment, the three SLA samples are chiral. While the chirality for both SLA-1 and SLA-2 is derived from the sugar and archaeol moieties, the chirality of SLA-3 only stems from the sugar moiety. To evaluate whether archaeol stereochemistry had any effect on the adjuvanticity of the glycolipids, the optical rotations of archaeols were first measured using a polarimeter. Table 1 shows the specific rotations of samples in solutions. As expected, the biological archaeol 1 produced the highest specific rotation (9.68), while optical activity was absent for the newly synthesized archaeol 3. A slightly lower specific rotation was found for the Avanti archaeol sample 2, in line with the lower stereoselectivity at the C2 center (94% of the R-form).

TABLE 1
Specific rotation and m/z value ([M + H]⁺) of archaeols

| Compound | Chirality | [α]_{D} | m/z exp. | m/z calc. |
|---|---|---|---|---|
| Archaeol (1) | 100% R | 9.68 | 653.6806 | 653.6806 |
| Archaeol (2) | 94% R | 9.38 | 653.6805 | 653.6806 |
| Archaeol (3) | Optically inactive | 0 | 653.6807 | 653.6806 |

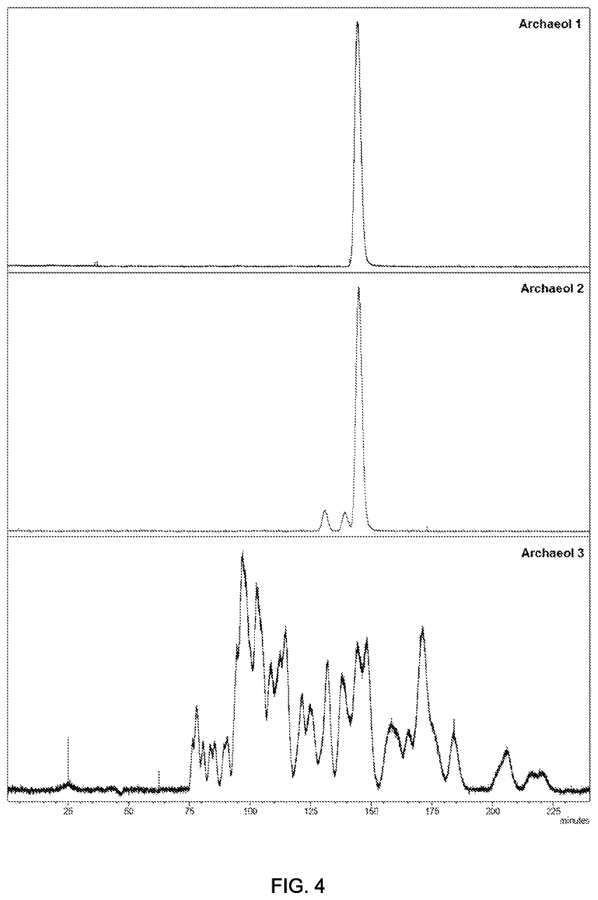

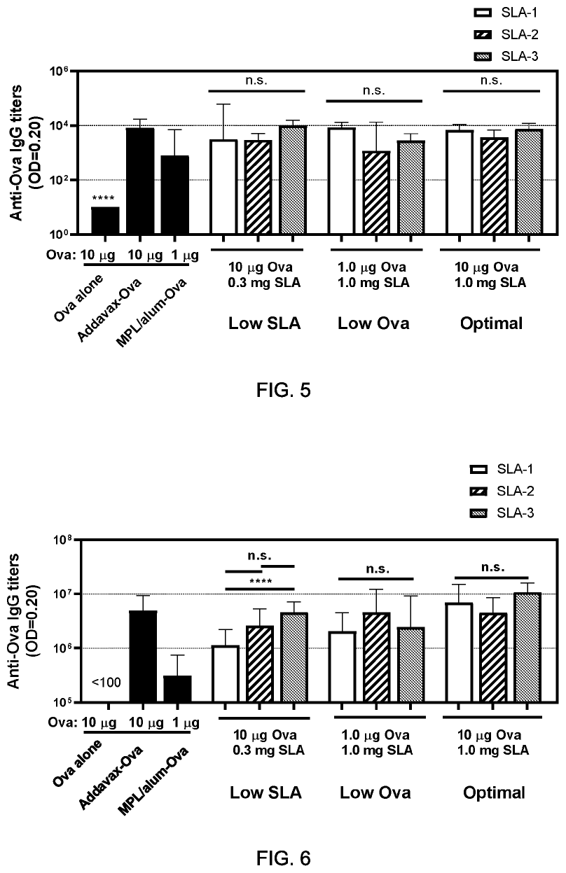

The measurement of specific rotation using a polarimeter is usually convenient and rapid, however this technique can only provide an approximate enantiomeric composition. Direct resolution of a racemate or enantioenriched sample using a chiral stationary phase (CSP) in high performance liquid chromatography (HPLC) has evolved into a reliable analytical method for the determination of enantiomeric excess of chiral compounds or the resolution of different components. Since enantiomers interact differently with the CSP during HPLC, the isomers elute at different speeds, resulting into separation.²³ Diastereomers have different properties and although in theory standard laboratory techniques, e.g., thin layer chromatography (TLC), can be used to identify and separate them, it is not always possible to separate the different isomers using these regular chromatography techniques. In this case, the three archaeol samples displayed one spot by TLC. Their exact mass was confirmed using high resolution mass spectrometry (HRMS) in combination with a C8 reverse-phase chromatography (Table 1). Using electrospray ionization in the positive mode, a same unique peak was observed for all archaeols at retention time (r.t.) of 4.3 min, with all mass spectra showing molecular ion [M+H]Y at m/z 653.6806, 653.6805 and 653.6807 (mass error 0.0/0.2/0.0 ppm) for samples 1, 2 and 3, respectively (m z calc. for C₄₃H₈₉O₃[M+H]⁺: 653.6806), ammonium adduct [M+NH₄]⁺ at m/z 670.7172, and a fragment at m/z 373.3676 corresponding to the loss of one phytyl unit. No chiral separation was obtained in the absence of the CSP as both the R and S isomers co-elute. A chiral column (Lux i-Amylose 3-Phenomenex Inc.) was thus used in combination with MS to resolve the various isomers of the archaeol samples (FIG. 4). For the biological archaeol 1, extracted ion chromatogram (m/z 653.65) showed only one peak at r.t. 144.8 min, with no visible peak splitting, shouldering or broadening, thus confirming the high optical purity of archaeol 1 (100% of (R)-2,3-bis(((3R,7R,11R)-3,7,11,15-tetramethylhexadecyl)oxy) propan-1-ol). On the other hand, the chromatogram corresponding to Avanti's archaeol 2 led to the separation of three peaks corresponding to the two epimers and an isomer of the R-form at the C2 center. Respective chiral HPLC purities were estimated to be 6% for (S)-2,3-bis(((3R,7R,11R)-3,7,11,15-tetramethylhexadecyl)oxy) propan-1-ol at r.t. 130.8 min; 94% for the total R configuration at the C2 with 88% of (R)-2,3-bis(((3R,7R,11R)-3,7,11,15-tetramethylhexadecyl)oxy) propan-1-ol at r.t. 144.8 min and 6% of (R)-2,3-bis(((3S,7R,11R)-3,7,11,15-tetramethylhexadecyl)oxy) propan-1-ol at r.t. 139.3 min where there is a different configuration, potentially at the C3 of the phytyl unit. The chiral HPLC purities are in line with the claimed optical purities. Chiral MS chromatography of archaeol 3 confirmed its highly complex chiral composition with more than 20 peaks showing the same molecular ion [M+H]⁺ at m/z 653.65. Unlike archaeol 2 where the stereoisomers arise mainly from the difference in spatial arrangement of the O-phytanyl moiety at the glycerol C-2, the optically inactive archaeol 3, with its 7 chiral centers, could have up to 128 enantiomer configurations (2⁷=128), which may explain the numerous peaks observed.

The fraction of archaeol 3 that is in the 100% R configuration (corresponding to (R)-2,3-bis(((3R,7R,11R)-3,7,11,15-tetramethylhexadecyl)oxy) propan-1-ol and also referred to as 100% R-form) was estimated using peak area. Archaeol-1 (100% R configuration) elutes at a retention time of 144.2 minutes. To determine the percentage of archaeol 3 that is equivalent to archaeol 1 (or 100% R configuration), the peak area of archaeol 3 at 144.2 minutes was determined and divided that by the total area for all peaks (see FIG. 9 and Table 2). Approximately 6.5% of archaeol 3 was found to have the 100% R configuration. It can therefore be extrapolated that while 100% of SLA contains 100% of archaeol 3, 100% of SLA-3 only contains about 6.5% of archaeol 1. The remaining 93.5% of SLA-3 contains other stereoisomers of archaeol.

TABLE 2
Calculation of the percentage of archaeol 3 that is in the 100% R
configuration

| | Total | Total | Peak | | % of (R)-2,3-bis(((3R,7R,11R)- |
|---|---|---|---|---|---|
| | Peak | Peak | Area (R) | | 3,7,11,15- |
| | Area | Area | RT: 144.2 | Total | tetramethylhexadecyl)oxy) |
| Ion m/z | before R | after R | min | Area | propan-1-ol (Archaeol 1) |
| 653.65 | 11689231 | 6161083 | 1232475 | 19082789 | 6.46% |

### Example 3: Assessment of SLA Adjuvanticity

### Materials and Methods

### Mice and Route of Immunization:

C57BL/6NCrl mice (6-8 weeks) were obtained from Charles River Laboratories (Saint-Constant, QC, Canada). Mice were maintained in individually ventilated cages with five female mice to a cage with easy access to food and water in a specific pathogen-free small animal facility with automatically controlled light/dark cycles, humidity and temperature at the National Research Council of Canada (NRC) in accordance with the guidelines of the Canadian Council on Animal Care. The animal use protocol (2016.08) was approved by the NRC Animal Care Committee. All mice were randomized upon entering the facility and were immunized and had samples collected and tested in a blinded method.

### Route of Immunization and Schedule:

C57BL/6NCrl mice were immunized by i.m. injection (50 μL) into the left tibialis anterior muscle. All mice were immunized twice in a prime/boost regime on day 0 and day 21. Blood samples were taken for sera collection before boost on day 20 as well as on day 28 immediately prior to euthanasia where whole spleens were collected in necropsy.

### Vaccine Preparation:

SLA archaeosomes were prepared as described previously.¹² Briefly, SLA lipids (SLA-1, SLA-2 or SLA-3) were dissolved in chloroform/methanol and aliquoted to a glass vial; the organic solvent was removed under N₂ gas with mild heating to form a thin lipid layer. A vacuum was applied for at least 2 h to ensure total removal of trace solvents. The lipid film was then hydrated with 1.0 mL of Milli-Q water and was shaken for 2 h at 40° C. or until hydration was completed. Archaeosome vesicles were reduced in size using a tabletop ultrasonic water bath (Fisher Scientific FS60H, 130 W and operating frequency of 40 kHz) and high pressure; they were then left to anneal at 4° C. for 12 h in static conditions and finally filter-sterilized through 0.22 μm filter units. The Ova protein solution (type VI, Sigma-Aldrich, Oakville, ON, Canada) was then added to the empty archaeosomes at the desired amount immediately before immunization so that a single dose contained 1 mg or 0.3 mg of SLA and 10 μg or 1 μg of antigen. The commercial adjuvant AddaVax™ (squalene-oil-in-water emulsion, Invivogen, San Diego, CA, USA) was prepared according to manufacturer's recommendations and mixed with 10 μg Ova protein at 1:1, v:v. The TLR4 agonist monophosphoryl Lipid A (MPLA from S. Minnesota R595 VacciGrade, InvivoGen, San Diego, CA, USA) was combined with Alum (Alhydrogel™ “85”, aluminum hydroxide, Brenntag Biosector, Frederikssund, Denmark), 10 μg: 40 μg respectively and 1 μg Ova. All solutions were brought to a physiological pH of 7.4 in phosphate-buffered saline (PBS).

### Assessment of IgG Titres:

Anti-Ova total IgG titers in mouse serum were quantified by ELISA as described previously.¹¹⁻¹² Briefly, 96-well high-binding ELISA plates (Thermo Fisher Scientific, Waltham, MA, USA) were coated overnight with 10 μg/mL of the Ova protein used for immunization. Plates were washed in 0.05% Tween20 in PBS (PBS-T; Sigma-Aldrich, Oakville, ON, Canada) and then blocked with 10% heat-inactivated bovine serum (Thermo Fisher Scientific, Waltham, MA, USA) in PBS for 1 h at 37° C. and washed again. Serum samples were 3.162-fold serially diluted in PBS-T, aliquoted to the plates and incubated for 1 h at 37° C. After washing, 1:4000 diluted secondary antibody, horseradish peroxidase conjugated goat anti-mouse IgG (Southern Biotech, Birmingham, AL, USA), was added to the plates. After washing, plates were developed with the substrate o-phenylenediamine dihydrochloride (OPD, Sigma-Aldrich, Oakville, ON, Canada) according to manufacturer's instructions. Titers for IgG in serum were defined as the dilution that resulted in an absorbance value (OD 450) of 0.2 and calculated using XLfit software (ID Business Solutions, Guildford, UK). No detectable titers were measured in serum samples from naïve control animals.

### Enumeration of Interferon-Gamma (IFN-γ) Secreting Cells by ELISPOT:

The Enumeration of IFN-γ secreting cells was done by use of an ELISPOT assay as previously described.¹² Briefly, spleen cells (at a final cell density of 4×10⁵ cells/well) were added to 96-well ELISPOT plates coated with anti-IFN-γ (Mabtech Inc., Cincinnati, OH, USA), and incubated in the presence of a peptide stimulant (or non-stimulant control) for 20 h at 37° C., 5% CO₂. Peptide stimulant consisted of SIINFEKL, an Ovalbumin CD8⁺ T cell epitope Ova257-264. Plates were then incubated, washed and developed using AEC substrate (Becton Dickinson, Franklin Lakes, NJ, USA) and counted using an automated ELISPOT plate reader by BIOSYS (Miami, FL, USA).

### In Vivo Cytotoxicity Assay:

The activity of antigen-specific CD8⁺ T cells (CTL) was measured in vivo as previously described.¹⁰ Briefly, donor spleen-cell suspensions from naïve C57BL/6NCrl mice were prepared. Cells were split into two aliquots. One aliquot was incubated at 37° C. with 10 g SIINFEKL (JPT Peptide Technologies GmbH) in RIO complete medium (RPMI 1640 with 10% heat-inactivated fetal bovine serum, 1% penicillin/streptomycin, 1% glutamine and 55 μM 2-mercaptoethanol) (Thermo Fisher Scientific, Waltham, MA, USA). After 30 min of incubation, the peptide-pulsed aliquot was stained with a high 10× concentration of CFSE (2.5 μM; Thermo Fisher Scientific); the second non-peptide-pulsed aliquot was stained with 1×CFSE (0.25 M). Two aliquots of cells (10×10⁶/each) were mixed 1:1 and injected into previously immunized recipient mice. Mice injected with Ova alone dissolved in PBS served as controls. At ˜20 to 22 h after the donor cell transfer, spleens were removed from recipients, single-cell suspensions prepared, and cells analyzed by flow cytometry on a BD Fortessa flow cytometer (Becton Dickinson, Franklin Lakes, NJ, USA). The in vivo percentage of peptide pulsed targets relative to non-peptide pulsed targets was enumerated according to a previously published equation.^{21, 24-25}

### Statistical Analysis:

Data were analyzed using GraphPad Prism 9® (GraphPad Software, San Diego, CA, USA). Antibody titers were log transformed prior to testing and shown as geometric mean titer (GMT) with the lower and upper 95% confidence interval shown in the graph and written in the text. All in vivo generated data were analyzed with the one-way analysis of variance (ANOVA) test followed by post-hoc analysis using Tukey's multiple comparisons test (comparisons among all groups), as indicated in the figure legends. For all analyses, differences were considered to be not significant with p>0.05.

### Results and Discussion

With three SLA samples in hand (SLA-1, SLA-2 and SLA-3), three different archaeosome formulations were produced and thereafter the impact of optical differences on the ability of archaeosomes to induce antigen-specific immune responses was investigated. SLA archaeosomes based on the natural 100% R-form archaeol have been previously shown to induce potent antigen-specific humoral and cellular immune responses when co-delivered with antigen.¹⁴ Therefore, the two fully synthetic SLA formulations, SLA-2 (synthesized from 94% R-form of synthetic archaeol) and SLA-3 (synthesized from non-stereoselective synthetic archaeol) were compared to the traditionally produced semi-synthetic formulation, SLA-1 (synthesized from 100% R-form of biological archaeol) and their ability to induce antigen-specific immune responses was measured following immunization of C57BL/6NCrl mice on days 0 and 21. Full-length Ova protein was mixed (10 μg/injection) with pre-formed empty archaeosomes (1 mg/injection) on the day of immunization. Controls include an unadjuvanted Ova group as well as Ova adjuvanted with mimetics of the approved adjuvants, AS04 and MF59, i.e., MPL/alum and AddaVax™, respectively. Antigen and adjuvant doses were selected based on previous experience in the inventors' laboratories as well as manufacturer's recommendations. Since immune responses may be saturated when using optimized antigen/adjuvant doses, lower doses of SLA archaeosomes (0.3 mg/injection) or Ova antigen (1 μg/injection) were also tested to better enable detection of any differences in immune responses following delivery of different SLA formulations. Serum was taken on days 20 and 28, to assess anti-Ova IgG responses following one or two immunizations respectively. On day 20, Ova-specific IgG titres were significantly enhanced for all groups when compared to immunization with Ova protein alone. As expected, a second immunization increased Ova-specific IgG titres from 10⁴ to 10⁶-10⁷ for most groups. Overall, there was little difference between the three different SLA formulations after either one or two immunizations (FIGS. 3 and 4, respectively) and responses obtained with all three SLAs were equivalent or better than those obtained using the two control adjuvants, MPL/alum and AddaVax™.

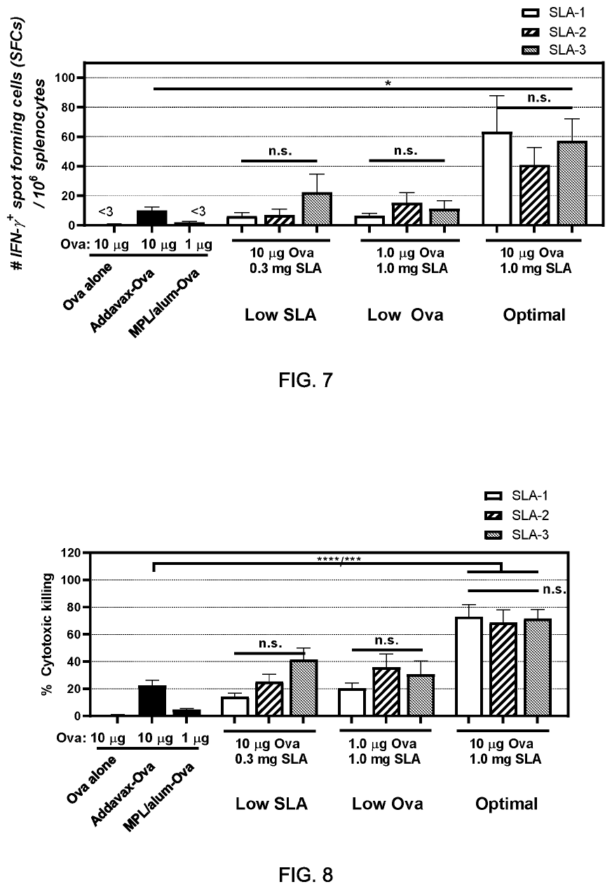

SLA archaeosomes are known for their ability to induce not only strong humoral, but also strong cell-mediated antigen-specific immune responses.¹⁴ To assess vaccine-induced cellular responses, antigen-specific IFN γ-producing CD8⁺ T cells were enumerated in the spleens of immunized mice 7 days post-boost in an ELISPOT assay. Mice immunized with Ova antigen alone had less than three detectable spots, the lower threshold set for the assay, as did mice immunized with the manufacturer's recommended dose of MPL/alum (10 μg/40 μg) and 1 μg of Ova. The greatest number of IFN-γ spot-forming cells (SFCs) were observed in mice immunized with 1 mg SLA and 10 μg Ova, (40-60 SFCs/10⁶ splenocytes) with no statistically significant differences observed between the three SLA formulations (FIG. 7). A significant reduction in SFCs (6-15 SFCs/10⁶ splenocytes) was observed in mice immunized with a suboptimal antigen dose of 1 μg Ova (6-15 SFCs/10⁶ splenocytes) as well as in mice immunized with a suboptimal adjuvant dose of 0.3 mg SLA (6-22 SFCs/10⁶ splenocytes). Mice immunized with the manufacturer's recommended dose of AddaVax™ and 10 μg of Ova had on average 10 SFC's, which was significantly less than those of mice immunized with an optimal dose of SLA and Ova (1 mg and 10 g, respectively) (FIG. 7). Overall, at each dose tested, there were no statistically significant differences between SLA formulations (SLA-1, SLA-2 and SLA-3) to produce IFN-γ⁺ Ova-CD8⁺ T cells.

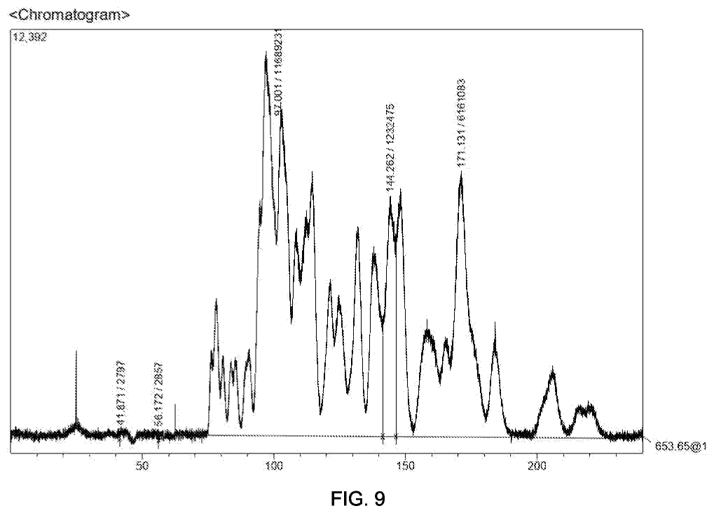

To assess the functionality of vaccine-induced antigen-specific CD8⁺ T cell responses in vaccinated mice, an in vivo cytotoxicity assay was conducted. The highest cytotoxic responses were observed in mice immunized with an optimal dose of SLA and Ova (1 mg and 10 μg respectively) and no differences were observed between the three different SLA formulations. Similarly, no differences between SLA formulations were observed at both the suboptimal SLA dose of 0.3 mg as well as at the suboptimal antigen dose of 1 μg Ova (FIG. 8). Similar to the ELISPOT results, a lower Ova antigen or SLA adjuvant dose resulted in lower CD8⁺ T cell cytotoxicity for all three formulations, SLA-1, SLA-2 and SLA-3. When SLA was compared to the commercial adjuvant AddaVax™ at the same antigen dose, all three SLA formulations induced a significantly greater cytotoxic responses. Overall these results confirm the ELISPOT findings that CD8⁺ T cell responses induced by all three SLA formulations were comparable.

When comparing “optimal” SLA levels to “low” SLA levels, it is apparent that reducing the amount of SLA-1 from 1.0 mg to 0.3 mg significantly reduces the ability of SLA-1 to induce production of IFN-γ⁺ Ova-CD8⁺ T cells (FIG. 7) and cytotoxic killing (FIG. 8). Lesser reductions were observed when comparing “low” SLA-3 (0.3 mg) versus “optimal” SLA-3 (1.0 mg), although there was no statistically significant difference between the results observed for SLA-3 compared to the results observed for SLA-1, perhaps due to small sample size. The significant reduction in the ability to induce production of IFN-γ⁺ Ova-CD8⁺ T cells or cytotoxic killing observed for SLA-1 when comparing 1.0 mg SLA-1 to 0.3 mg SLA-1 (i.e. 30% of the original amount of SLA-1) indicates that stereoisomers within the SLA-3 mixture, other than the 100% R-form, are able to induce immune responses, as only about 6.5% of SLA-3 is of the 100% R-form configuration (equivalent to 0.065 mg of SLA-1). The results shown in FIGS. 7 and 8 suggest that SLA-3 is at least as effective as SLA-1, and might be even more effective than SLA-1.

The preceding examples have been provided to allow a greater understanding of the present disclosure by illustrating specific examples that are in accordance with embodiments of the disclosure. The accompanying claims should not be limited to the specific details provided in the examples. Rather, they should be given the broadest interpretation that is consistent with the collective teaching of the specification and drawings, in consideration of the common general knowledge in the art.

### REFERENCES

1. Patel, G. B.; Sprott, G. D., Archaeobacterial Ether Lipid Liposomes (Archaeosomes) as Novel Vaccine and Drug Delivery Systems. Critical Reviews in Biotechnology 1999, 19 (4), 317-357.
2. Kaur, G.; Garg, T.; Rath, G.; Goyal, A. K., Archaeosomes: an excellent carrier for drug and cell delivery. Drug Delivery 2016, 23 (7), 2497-2512.
3. Agbayani, G.; Jia, Y.; Akache, B.; Chandan, V.; Iqbal, U.; Stark, F. C.; Deschatelets, L.; Lam, E.; Hemraz, U. D.; Régnier, S.; Krishnan, L.; McCluskie, M. J., Mechanistic insight into the induction of cellular immune responses by encapsulated and admixed archaeosome-based vaccine formulations. Human Vaccines & Immunotherapeutics 2020, 16 (9), 2183-2195.
4. Aoki, T.; Poulter, C. D., Archaebacterial isoprenoids. Synthesis of 2,3-di-O-phytanyl-sn-glycerol and its 1,2-isomer. The Journal of Organic Chemistry 1985, 50 (26), 5634-5636.
5. Smith, S. W., Chiral Toxicology: It's the Same Thing . . . Only Different. Toxicological Sciences 2009, 110 (1), 4-30.
6. Franks, M. E.; Macpherson, G. R.; Figg, W. D., Thalidomide. The Lancet 2004, 363 (9423), 1802-1811.
7. Ameredes, B. T.; Calhoun, W. J., Levalbuterol versus albuterol. Current Allergy and Asthma Reports 2009, 9 (5), 401-409.
8. Pashine, A.; Valiante, N. M.; Ulmer, J. B., Targeting the innate immune response with improved vaccine adjuvants. Nature Medicine 2005, 11 (S4), S63-S68.
9. Conlan, J. W.; Krishnan, L.; Willick, G. E.; Patel, G. B.; Sprott, G. D., Immunization of mice with lipopeptide antigens encapsulated in novel liposomes prepared from the polar lipids of various Archaeobacteria elicits rapid and prolonged specific protective immunity against infection with the facultative intracellular pathogen, Listeria monocytogenes. Vaccine 2001, 19 (25-26), 3509-3517.
10. Krishnan, L.; Deschatelets, L.; Stark, F. C.; Gurani, K.; Sprott, G. D., Archaeosome Adjuvant Overcomes Tolerance to Tumor-Associated Melanoma Antigens Inducing Protective CD8+ T Cell Responses. Clinical and Developmental Immunology 2010, 2010, 1-13.
11. Akache, B.; Stark, F. C.; Jia, Y.; Deschatelets, L.; Dudani, R.; Harrison, B. A.; Agbayani, G.; Williams, D.; Jamshidi, M. P.; Krishnan, L.; McCluskie, M. J., Sulfated archaeol glycolipids: Comparison with other immunological adjuvants in mice. Plos One 2018, 13 (12).
12. Jia, Y.; Akache, B.; Deschatelets, L.; Qian, H.; Dudani, R.; Harrison, B. A.; Stark, F. C.; Chandan, V.; Jamshidi, M. P.; Krishnan, L.; McCluskie, M. J., A comparison of the immune responses induced by antigens in three different archaeosome-based vaccine formulations. International Journal of Pharmaceutics 2019, 561, 187-196.
13. Stark, F. C.; Akache, B.; Ponce, A.; Dudani, R.; Deschatelets, L.; Jia, Y.; Sauvageau, J.; Williams, D.; Jamshidi, M. P.; Agbayani, G.; Wachholz, K.; Harrison, B. A.; Li, X.; Krishnan, L.; Chen, W.; McCluskie, M. J., Archaeal glycolipid adjuvanted vaccines induce strong influenza-specific immune responses through direct immunization in young and aged mice or through passive maternal immunization. Vaccine 2019, 37 (47), 7108-7116.
14. Jia, Y.; Akache, B.; Agbayani, G.; Chandan, V.; Dudani, R.; Harrison, B. A.; Deschatelets, L.; Hemraz, U. D.; Lam, E.; Régnier, S.; Stark, F. C.; Krishnan, L.; McCluskie, M. J., The Synergistic Effects of Sulfated Lactosyl Archaeol Archaeosomes When Combined with Different Adjuvants in a Murine Model. Pharmaceutics 2021, 13 (2).
15. Akache, B.; Agbayani, G.; Stark, F. C.; Jia, Y.; Dudani, R.; Harrison, B. A.; Deschatelets, L.; Chandan, V.; Lam, E.; Hemraz, U. D.; Régnier, S.; Krishnan, L.; McCluskie, M. J., Sulfated Lactosyl Archaeol Archaeosomes Synergize with Poly(I:C) to Enhance the Immunogenicity and Efficacy of a Synthetic Long Peptide-Based Vaccine in a Melanoma Tumor Model. Pharmaceutics 2021, 13 (2).
16. Whitfield, D. M.; Yu, S. H.; Dicaire, C. J.; Sprott, G. D., Development of new glycosylation methodologies for the synthesis of archaeal-derived glycolipid adjuvants. Carbohydrate Research 2010, 345 (2), 214-229.
17. Sprott, G. D.; Yeung, A.; Dicaire, C. J.; Yu, S. H.; Whitfield, D. M., Synthetic Archaeosome Vaccines Containing Triglycosylarchaeols Can Provide Additive and Long-Lasting Immune Responses That Are Enhanced by Archaetidylserine. Archaea 2012, 1-9.
18. Whitfield, D. M. S., G. D.; Krishnan, L. Sulfated-glycolipids as adjuvants for vaccines. 2016.
19. Golding, B. T., and P. V. Ioannou, Rapid syntheses of 3-0-benzyl-sn-glycerol and 2-0-benzylglycerol. Synthesis 1977, 423-424.
20. Lucquin, A.; Colonese, A. C.; Farrell, T. F. G.; Craig, O. E., Utilising phytanic acid diastereomers for the characterisation of archaeological lipid residues in pottery samples. Tetrahedron Letters 2016, 57 (6), 703-707.
21. Konradsson, P.; Mootoo, D. R.; McDevitt, R. E.; Fraser-Reid, B., Iodonium ion generated in situ from N-iodosuccinimide and trifluoromethanesulphonic acid promotes direct linkage of ‘disarmed’ pent-4-enyl glycosides. J. Chem. Soc., Chem. Commun. 1990, (3), 270-272.
22. Cook, C.; Byrne, S.; d'Aubigny, C. D.; Viola, D.; Mikucki, J.; Ellis, W., Detection Limits for Chiral Amino Acids Using a Polarization Camera. The Planetary Science Journal 2020, 1 (2).
23. Okamoto, Y.; Ikai, T., Chiral HPLC for efficient resolution of enantiomers. Chemical Society Reviews 2008, 37 (12).
24. Barber, D. L.; Wherry, E. J.; Ahmed, R., Cutting Edge: Rapid In Vivo Killing by Memory CD8 T Cells. The Journal of Immunology 2003, 171 (1), 27-31.
25. Stark, F. C.; Dudani, R.; Agbayani, G.; McCluskie, M. J., A Method to Evaluate In Vivo CD8+ T Cell Cytotoxicity in a Murine Model. In Vaccine Delivery Technology, 2021; pp 549-558.

What is claimed is:

1. A composition comprising a mixture of two or more stereoisomers of a synthetic charged isoprenoid glycolipid comprising a sulfated saccharide group covalently linked to the free sn-1 hydroxyl group of the glycerol backbone of an archaeol moiety via a beta linkage, wherein the synthetic charged glycolipid is a compound of the formula: 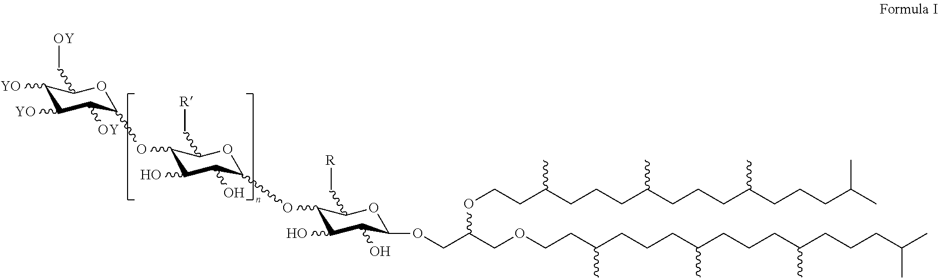
or a pharmaceutically acceptable salt thereof,
wherein
n is 0 or 1;
R and R′ are independently hydrogen or hydroxyl;
each Y is independently hydrogen or a sulfate group, and wherein at least one Y is a sulfate group; and
less than 25% of the synthetic charged isoprenoid glycolipid molecules in the mixture comprise an archaeol moiety of the configuration (R)-2,3-bis(((3R,7R,11R)-3,7,11,15-tetramethylhexadecyl)oxy) propan-1-ol.

2. The composition of claim 1, wherein only one Y is a sulfate group.

3. The composition of claim 1 or 2, wherein the sulfated saccharide group comprises monosaccharide moieties selected from the group consisting of mannose (Man), glucose (Glc), rhamnose (Rha), and galactose (Gal) moieties.

4. The composition of claim 3, wherein the compound comprises a sulfate group at the 6′ position of the terminal monosaccharide moiety.

5. The composition of any one of claims 1 to 4, wherein n is 0 and R is OH.

6. The composition of any one of claims 1 to 5, wherein the compound is 6′-sulfate-α-D-Manₚ-(1,6)-β-D-Galₚ-(1,4)-β-D-Glcₚ-(1,1)-archaeol, or 6′-sulfate-β-D-Glcₚ-(1,6)-β-D-Galₚ-(1,4)-β-D-Glcₚ-(1,1)-archaeol, or 6′-sulfate-β-D-Galₚ-(1,4)-β-D-Glcₚ-(1,6)-β-D-Glcₚ-(1,1)-archaeol.

7. The composition of any one of claims 1 to 6, wherein the sulfated saccharide group is a sulfated lactosyl group.

8. The composition of claim 7, wherein the sulfated lactosyl group is a 6′-S-lactosyl group.

9. The composition of claim 8, wherein the 6′-S-lactosyl group is 6′-sulfate-β-D-Galₚ-(1,4)-β-D-Glcₚ.

10. The composition of any one of claims 1 to 9 wherein the synthetic charged glycolipid is a compound of the structure: 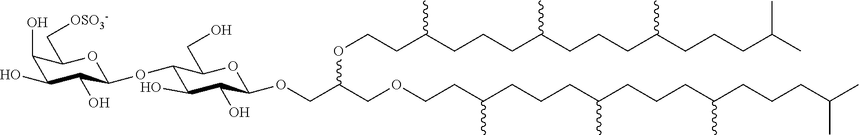
or a pharmaceutically acceptable salt thereof.

11. The composition of any one of claims 1 to 10, wherein less than 10% of the synthetic charged isoprenoid glycolipid molecules in the mixture comprise an archaeol moiety of the configuration (R)-2,3-bis(((3R,7R,11R)-3,7,11,15-tetramethylhexadecyl)oxy) propan-1-ol.

12. The composition of claim 11, wherein about 5% to about 8% of the synthetic charged isoprenoid glycolipid molecules in the mixture comprise an archaeol moiety of the configuration (R)-2,3-bis(((3R,7R,11R)-3,7,11,15-tetramethylhexadecyl)oxy) propan-1-ol.

13. The composition of claim 12, wherein about 6.5% of the synthetic charged isoprenoid glycolipid molecules in the mixture comprise an archaeol moiety of the configuration (R)-2,3-bis(((3R,7R,11R)-3,7,11,15-tetramethylhexadecyl)oxy) propan-1-ol.

14. An archaeosome comprising the composition of any one of claims 1 to 13.

15. An immunogenic composition comprising the composition of any one of claims 1 to 13 or the archaeosome of claim 14, and an antigen.

16. The immunogenic composition of claim 15, wherein the antigen is a peptide, protein, or virus-like particle.

17. The immunogenic composition of claim 15 or 16, further comprising a pharmaceutically acceptable carrier.

18. The immunogenic composition of any one of claims 15 to 17, further comprising an adjuvant other than a synthetic charged isoprenoid glycolipid.

19. The immunogenic composition of any one of claims 15 to 18, wherein the immunogenic composition is a vaccine composition.

20. A method of inducing an immune response in a subject, the method comprising administering the immunogenic composition of any one of claims 15 to 19 to a subject.

21. A process for synthesizing a composition comprising a mixture of two or more stereoisomers of an archaeol, the process comprising treating (±)-3-benzyloxy-1,2-propanediol with a mesylated phytol derivative through a double nucleophilic substitution reaction, followed by a reductive debenzylation reaction,
wherein the archaeol is of the structure: 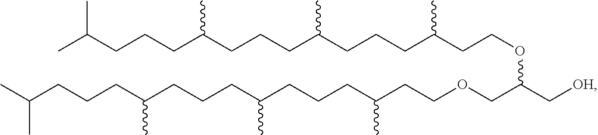
and wherein the mesylated phytol derivative is of the structure:

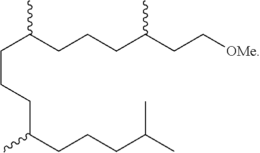

22. The process of claim 21, wherein the process comprises the following steps:

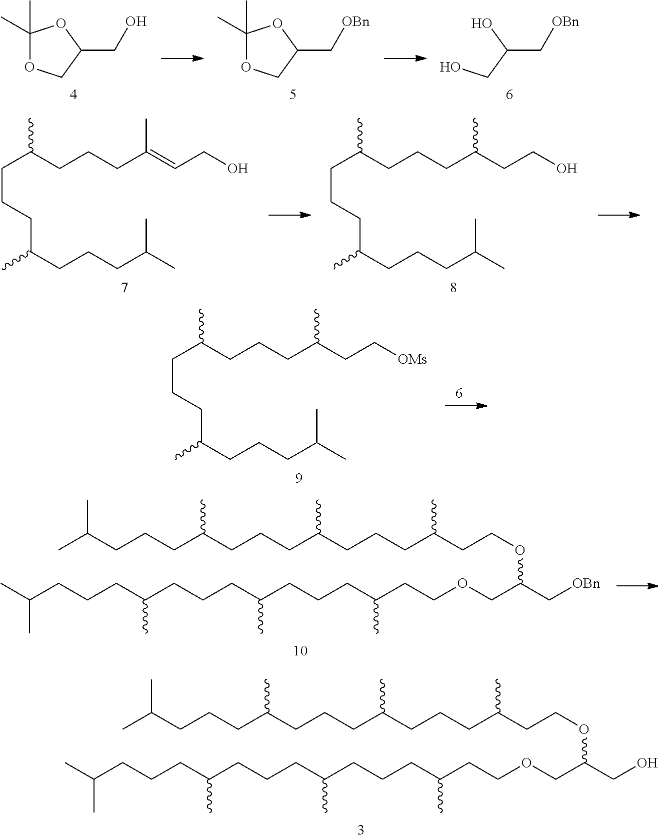

23. A process for synthesizing a mixture of two or more stereoisomers of a synthetic charged isoprenoid glycolipid or a pharmaceutically acceptable salt thereof, the process comprising covalently linking a sulfated saccharide group of the formula: 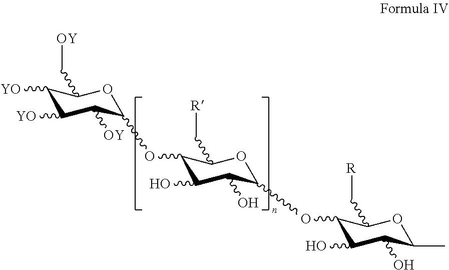
to the free sn-1 hydroxyl group of the glycerol backbone of an archaeol,
wherein the archaeol comprises a mixture of two or more stereoisomers of the structure: 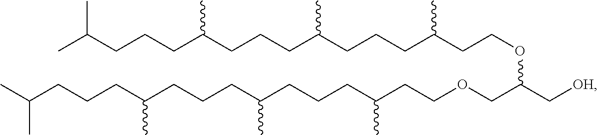
and wherein
n is 0 or 1;
R and R′ are independently hydrogen or hydroxyl;
each Y is independently hydrogen or a sulfate group, and wherein at least one Y is a sulfate group; and
less than 25% of the archaeol molecules in the mixture of two or more stereoisomers are of the configuration (R)-2,3-bis(((3R,7R,11R)-3,7,11,15-tetramethylhexadecyl)oxy) propan-1-ol.

24. The process of claim 23, wherein the saccharide group is of the formula:

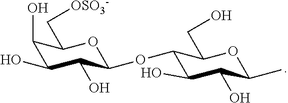

25. The process of claim 23 or 24, wherein less than 10% of the archaeol molecules in the mixture of two or more stereoisomers are of the configuration (R)-2,3-bis(((3R,7R,11R)-3,7,11,15-tetramethylhexadecyl)oxy) propan-1-ol.

26. The process of claim 23 or 24, wherein about 5% to about 8% of the archaeol molecules in the mixture of two or more stereoisomers are of the configuration (R)-2,3-bis(((3R,7R,11R)-3,7,11,15-tetramethylhexadecyl)oxy) propan-1-ol.

27. The process of claim 23 or 24, wherein about 6.5% of the archaeol molecules in the mixture of two or more stereoisomers are of the configuration (R)-2,3-bis(((3R,7R,11R)-3,7,11,15-tetramethylhexadecyl)oxy) propan-1-ol.

28. The process of any one of claims 23 to 27, wherein the archaeol is produced according to the process defined in claim 21 or 22.

29. Use of the composition of any one of claims 1 to 13 or the archaeosome of claim 14 for the manufacture of a vaccine or immunogenic composition.

30. Use of the composition of any one of claims 1 to 13 or the archaeosome of claim 14 as an adjuvant in a vaccine or immunogenic composition.

31. Use of the composition of any one of claims 1 to 13 or the archaeosome of claim 14 as an adjuvant to enhance or direct an immune response to an antigen in a subject.

32. The composition of any one of claims 1 to 13 or the archaeosome of claim 14, for use to enhance or direct an immune response to an antigen in a subject.

33. Use of the immunogenic composition of any one of claims 15 to 19 to induce an immune response to the antigen in a subject.
